(19) United States
(12) Patent Application Publication
(10) Pub. No.: US 2020/0376036 A1
(43) Pub. Date: Dec. 3, 2020

(54) METHODS OF REGULATORY T CELL EXPANSION AND ACTIVATION

(71) Applicant: University of Miami, Miami, FL (US)

(72) Inventors: Robert LEVY, Miami, FL (US); Sabrina COPSEL, Miami, FL (US); Dietlinde WOLF, Miami, FL (US); Krishna KOMANDURI, Miami, FL (US)

(21) Appl. No.: 16/970,792

(22) PCT Filed: Feb. 20, 2019
(86) PCT No.: PCT/US19/18660

§ 371 Date: Aug. 18, 2020

Related U.S. Application Data
(60) Provisional application No. 62/632,827, filed on Feb. 20, 2018.

(51) Int. Cl. A61K 35/28; C07K 14/525; C07K 16/28; A61P 37/06

(52) U.S. Cl. CPC A61K 35/28; C07K 14/525; A61K 38/2013; A61P 37/06; C07K 16/2878

(57) ABSTRACT

The present invention relates to methods for modulating regulatory T cells (Tregs) for use, e.g., in treating or preventing graft-versus-host disease (GVHD) in a transplant recipient.

### CROSS-REFERENCE TO RELATED APPLICATIONS

This application claims priority to and the benefit of U.S. Provisional Patent Application No. 62/632,827, filed on Feb. 20, 2018, the entire content of which is hereby incorporated herein by reference in its entirety.

### FIELD

The present invention relates to methods for modulating regulatory T cells (Tregs) for use, e.g., in autoimmune diseases or disorders, and in prevention and treatment of organ transplant rejection.

### SEQUENCE LISTING

This application contains a Sequence Listing which has been submitted in ASCII format via EFS-Web and is hereby incorporated by reference in its entirety. Said ASCII copy, created on Feb. 14, 2019, is named PEL-013PC_SEQUENCELISTING_ST25.txt and is 9,656 bytes in size.

### BACKGROUND

Tregs are T cells which have a role in regulating or suppressing other cells in the immune system. Stimulation of tumor necrosis factor receptor superfamily, member 25 (TNFRSF25) in vivo with its natural ligand tumor necrosis factor (TNF)-like cytokine 1A (TL1A, also known as TNFSF15), facilitates selective proliferation of Tregs in mice and suppression of immunopathology in allergic lung inflammation, allogeneic heart transplantation and HSV-1 mediated ocular inflammation. Progress in translating Treg therapy in humans has been slow and not without drawbacks such as, e.g., side effects and increase in inflammation. Although adoptive transfer of expanded Tregs can be useful in various autoimmune diseases, ex vivo generation of large numbers of functional Tregs remains difficult. Also, improved therapies that are safe and effective for Treg therapy in humans are needed.

### SUMMARY

Accordingly, the present invention relates to compositions and methods that are useful in modulating Tregs for use in, for instance, in vivo and ex vivo therapies for various autoimmune diseases or disorders and for prevention or treatment of organ transplant rejection.

In some aspects, the present invention relates to generation of Tregs by contacting a population of Tregs with an agent that targets tumor necrosis factor receptor superfamily member 25 (TNFRSF25), such as a TL1A-Ig fusion protein as described herein. In some embodiments, the population of Tregs can be additionally contacted with interleukin (e.g., low dose IL-2). The contacting leads, in vivo or ex vivo, to a markedly expanded and selectively activated Treg population. Moreover, the described methods include administering the fusion protein such that it can cause a sustained increase in Treg cells in a transplant recipient, which can be a solid organ transplant recipient.

Tregs, generated either in vivo or ex vivo, demonstrate phenotypic and functional differences from unexpanded Treg that make them better suited for use in preventing, reducing or ameliorating various autoimmune diseases or disorders. In various embodiments, the treated Treg of the present invention find use in treating or preventing GVHD in a transplant recipient. In various embodiments, the generated Tregs of the present invention find use in preventing transplant rejections, e.g., solid organ rejections (e.g., prior to the subject receiving the transplant). In various embodiments, the generated Tregs of the present invention find use in delaying acute rejection of a transplanted allograft in a subject (e.g., prior to the subject receiving the transplanted allograft).

In some aspects, a method of treating or preventing graft-versus-host disease (GVHD) in a transplant recipient is provided that comprises administering a human TL1A-Ig fusion protein to a transplant donor. The fusion protein comprises (a) a first polypeptide comprising an extracellular domain of a human TL1A polypeptide or a fragment thereof that specifically binds to TNFRSF25), and (b) a second polypeptide comprising an immunoglobulin (Ig) polypeptide. In the described method, the administration occurs at least 3 times, and the transplant comprises Tregs from the transplant donor. In some embodiments, the first polypeptide comprises the amino acid sequence of SEQ ID NO: 1, or an amino acid sequence that has at least 95% sequence identity to SEQ ID NO: 1. In some embodiments, the Ig polypeptide comprises one or more of a hinge region, a CH2 domain, and a CH3 domain of an IgG polypeptide. In some embodiments, the Ig polypeptide comprises the amino acid sequence of SEQ ID NO: 2, or an amino acid sequence that has at least 95% sequence identity to SEQ ID NO: 2. In some embodiments, the TL1A-Ig fusion protein has at least 95% sequence identity to the amino acid sequence of SEQ ID NO: 3.

In some aspects, a method of reducing or preventing an immune response in a solid organ transplant recipient is provided, which includes administering a human TL1A-Ig fusion protein to the solid organ transplant recipient, wherein the fusion protein comprises (a) a first polypeptide comprising an extracellular domain of a human TL1A polypeptide or a fragment thereof that specifically binds to TNFRSF25, and (b) a second polypeptide comprising an immunoglobulin (Ig) polypeptide. The administration occurs at least 3 times, and the immune response is a rejection of the solid organ transplant. The solid organ is selected from lung, kidney, heart, liver, pancreas, thymus, gastrointestinal tract, cornea, eye, and composite allografts. Composite allograft transplantation can be, for example, intestinal/multivisceral transplantation (which may or may not include liver) and its variants.

In some embodiments, a method for generating Tregs in vivo is provided that includes administering to a subject in need thereof a human TL1A-Ig fusion protein to expand and selectively activate a population of Treg. The fusion protein comprises (a) a first polypeptide comprising an extracellular domain of a human TL1A polypeptide or a fragment thereof that specifically binds to TNFRSF25, and (b) a second polypeptide comprising an immunoglobulin (Ig) polypeptide.

In some aspects, a method for generating Tregs ex vivo is provided that includes (a) isolating a population of Tregs from a subject, and (b) contacting the isolated population of Tregs with a human TL1A-Ig fusion protein, to expand and selectively activate a population of Tregs. The fusion protein comprises (a) a first polypeptide comprising an extracellular domain of a human TL1A polypeptide or a fragment thereof that specifically binds to TNFRSF25, and (b) a second polypeptide comprising an immunoglobulin (Ig) polypeptide.

In various embodiments, the generated Tregs find use in treating various autoimmune diseases or disorders, GVHD, preventing transplant rejections, delaying acute rejection of a transplanted allograft at lower doses than an untreated Tregs (e.g., 30% lower dose, 40% lower dose, 50% lower dose, 60% lower dose, 70% lower dose, 80% lower dose, 90% lower dose, 100% lower dose).

In various embodiments, the generated Tregs are characterized by significantly fewer naive Tregs and increased central memory (CD62-L^{hi}CD44+) and effector/memory (CD62-L^{lo}CD44+) Tregs, e.g., if in vivo, in the spleen and peripheral lymph nodes (pLN) as compared to untreated Tregs. Furthermore, in various embodiments, the generated Tregs exhibit a significant decrease in Ly6C expression as compared to untreated Tregs.

In some aspects, a method for preventing, reducing or ameliorating an autoimmune disease or disorder in a subject in need thereof is provided that comprises administering a human TL1A-Ig fusion protein, the fusion protein comprising (a) a first polypeptide comprising an extracellular domain of a human TL1A polypeptide or a fragment thereof that specifically binds to TNFRSF25, and (b) a second polypeptide comprising an immunoglobulin (Ig) polypeptide. In some aspects, the present invention provides a method for preventing, reducing or ameliorating an autoimmune disease or disorder in a subject in need thereof comprising administering a population of Tregs to the subject, wherein, the population of Tregs has been treated with the human TL1A-Ig fusion protein.

In various embodiments, higher levels (as compared to untreated Tregs) of activation and functional markers are present in the peripheral lymph nodes and the spleen in the present generation methods (when in vivo), such activation and functional markers including CD39, Nrp1, ICOS, CD73, KLRG1, CD103, Annexin V, PD-1 and CTLA-4.

In various embodiments, the generated Tregs of the present invention exhibit higher levels of Treg effector molecules (e.g., TGF beta, GzmB, GzmA, IFN gamma, and IL-10) and mediated enhanced in vitro suppressor activity as compared to unexpanded Tregs.

### BRIEF DESCRIPTION OF THE FIGURES

FIGS. 1A and 1B illustrate that a CD25 and TNFRSF25 “two-pathway” Tregs expansion affects FoxP3⁺CD4⁺Treg subset distribution. FIG. 1A shows the experimental design of the two-pathway system. FIG. 1B is a pair of flow cytometry histograms showing that in vivo treatment with TL1A-Ig+low dose IL-2 induced a strong increase in the overall levels of CD4⁺FoxP3⁺ within the CD4⁺ T cell compartment. FIG. 10 is a pair of flow cytometry graphs showing that in vivo treatment with TL1A-Ig+low dose IL-2 induced a strong increase in the overall levels of CD4⁺FoxP3⁺ within the CD4⁺ T cell compartment.

FIGS. 2A-D illustrate the two-pathway Treg expansion Treg subset distribution. FIG. 2A illustrates bar graphs showing that two-pathway expanded Tregs exhibited significantly fewer naive Tregs and increased central memory (CD62-L^{hi}CD44⁺) and effector/memory (CD62-L^{lo}CD44⁺) Tregs in the spleen as compared to untreated Tregs. FIG. 2B are bar graphs showing that significantly fewer naive Tregs and increased central memory (CD62-L^{hi}CD44⁺) and effector/memory (CD62-L^{lo}CD44⁺) Tregs in the peripheral lymph nodes (pLN) as compared to untreated Tregs. FIG. 2C and FIG. 2D are a pair of histograms and bar graphs, respectively, showing that expanded Tregs exhibited a significant decrease in Ly6C expression as compared to unexpanded Tregs.

FIGS. 3A-B are a series of bar graphs (FIG. 3A) and histograms (FIG. 3B) showing that in vivo expansion (via IL-2 and TL1A-Ig) change expression of activation/functional molecules in Tregs. FIG. 3A shows that higher levels of activation and functional markers (CD39, Nrp-1, ICOS, CD73, KLRG1. CD103, Annexin V, PD1, and CTLA-4) were observed in the peripheral lymph nodes between the expanded vs. unexpanded Tregs (similar results were observed in the spleen) and that increased expression of these molecules by Tregs is associated with differentiation, co-stimulation, DC engagement and migration. FIG. 3B are a series of histograms showing expanded and unexpanded expression of CD103, ICOS and Nrp-1 markers in effector Tregs, eTregs (CD62-L^{lo}Ly-6C⁻).

FIGS. 4A-B are a series of bar graphs showing that IL-2 and TL1A-Ig-driven in vivo expansion modifies Treg effector molecules and function and that two-pathway expanded Tregs exhibited higher mRNA levels of Treg effector molecules and mediated enhanced in vitro suppressor activity vs. unexpanded Tregs. FIG. 4A shows mRNA fold changes of TGF-β, GzmB, GzmA, IFN-y, and IL-10. FIG. 4B shows proliferation under no Tregs, unexpanded, and unexpanded Tregs.

FIGS. 5A-G show that two-pathway expanded Tregs mediate superior GVHD amelioration vs. unexpanded Tregs. FIG. 5A shows the experimental design of MHC-mismatched aHSCT (B6→BALB/c) mice performed using sorted expanded or unexpanded donor Tregs combined with T conventional cells. FIG. 5B shows the clinical score versus a number of days post hematopoietic stem cell transplantation (post-HSCT) and a survival plot of expanded, unexpanded, BM (control) only, and demonstrates that using 3.5×10⁵ unexpanded Tregs+1.0×10⁶ splenic T cells was not sufficient to prevent GVHD. In FIG. 5B, at day 28, the graphs are (from the top and down) GVHD, unexpanded Tregs (3.5×10⁵), expanded Tregs (1.75×10⁵), expanded Tregs (3.5×10⁵), and BM only. FIG. 5C shows the clinical score of days post-HSCT of expanded, unexpanded, BM (control) under lower Tregs conditions. Using as low as 1.75×10⁵ expanded CD4⁺FoxP3⁺ cells (ratio 0.2:1 Treg/Teff) was sufficient to diminish GVHD; however, lower numbers of expanded Tregs (0.05 to 1.0×10⁵) were not sufficient to prevent GVHD. FIG. 5D is a bar graph showing the percentage of donor Treg/CD4+ cells in the spleen and lymph nodes (mLN), 1-week post-transplant. FIG. 5E is a bar graph showing the percentage of donor Tcells in (⋄) GVHD, (●) unexpanded Tregs, (ϕ) expanded Tregs and (Δ) BM only, 4 weeks post-transplant; and an image showing mice treated with unexpanded Tregs, mice treated with expanded Tregs, and BM (control) mice. FIG. 5F is a picture and a bar graph showing the percentage of lymphocytes in (⋄) GVHD, (●) unexpanded Tregs, (ϕ) expanded Tregs, and (Δ) BM only, 5 weeks post-transplant. FIG. 5G is a pair of bar graphs showing CD4+ and CD8− engraftment under unexpanded-Tregs, expanded-Tregs and BM-only conditions, 5 weeks' post-transplant in (Top-bar graph) recipient, (Middle bar graph) T cell donor and (Bottom bar graph) BM donor cells.

FIGS. 6A and 6B are series of bar graphs showing that immune engraftment following MHC-mismatched HSCT is more rapid in recipients treated with expanded Tregs vs. plasmacytoid T cells (PTC). FIG. 6A shows the percentage of CD4 lymphocytes in expanded Tregs vs. PTC following days 30, 60 and 90 in BM donor (top bar graph), T cell donor (middle bar graph), and recipients (bottom bar graph). FIG. 6B shows the percentage of CD8 lymphocytes in expanded Tregs vs. PTC following days 30, 60 and 90 in BM donor (top bar graph), T cell donor (middle bar graph), and recipients (bottom bar graph).

FIGS. 7A-C are a series of bar graphs and histograms showing a less thymic damage in recipients of Treg expanded vs. PTC early post-HSCT. FIG. 7A shows the number of cells and percentage of total thymocytes following GVHD-control, PTC and expanded Treg treatments. FIG. 7B shows CD8 and CD4 cells following GVHD-control, PTC and Treg treatments. FIG. 7C shows the percentage of CD4⁺ and CD8⁺ lymphocytes following GVHD-control, PTC and expanded Treg treatment in BM donor (top bar graph), T cell donor (middle bar graph), and recipients (bottom bar graph).

FIGS. 8A-C are a series of bar graphs and histograms showing a Recent thymic emigrant (RTE) analysis. FIG. 8A is a schematic showing RTE analysis of PTC vs donor expanded Tregs in MHC mismatched HSCT. FIG. 8B and 8C show that higher levels of RTE present following MHC-mismatched HSCT using donor cells containing expanded Tregs vs normal donor cells and PTC treatment.

FIGS. 9A-C are a schematic and a series of graphs showing a TL1A-Ig+IL-2 protocol to monitor Treg activity. FIG. 9A is a schematic showing the 2 Week TL1A-Ig+IL-2 experimental design in Foxp3-IRES-mRFP (FIR) reporter mouse to monitor Treg activity. FIG. 9B shows the percentage of Foxp3⁺ CD4+ cells within total CD4⁺ cells in blood following mIgG1, TL1A-Ig and TL1A-Ig+IL-2 treatment in a time dependent manner (days 1, 5, 9, and 15) post injection. FIG. 9C shows the percentage of Foxp3⁺ CD4+ cells within total CD4⁺ cells following mIgG1, TL1A-Ig and TL1A-Ig+IL-2 treatment in lymph nodes and spleen on day 15.

FIG. 9D shows the cell number on day 15 following aCD3 3 days stimulation under mIgG1, TL1A-Ig, and TL1A-Ig+IL-2 treatments in the lymph nodes and spleen (200.000 cells in 200 μl were plated (1 million cells/ml), and the aCD3 final concentration was 1 μg/ml).

FIG. 9E shows the cell number on day 15 following LPS 4 days stimulation under mIgG1, TL1A-Ig and TL1A-Ig+IL-2 treatment in the lymph nodes and spleen (200.000 cells in 200 μl were plated (1 million cells/mi), and the LPS final concentration was 2 μg/ml).

FIG. 9F shows the total blood cells number on day 15 following mIgG1, TL1A-Ig, and TL1A-Ig+IL-2 treatments.

FIG. 9G shows the blood cells number following CD3, CD4, and Foxp3⁺ cells stimulation under mIgG1, TL1A-Ig, and TL1A-Ig+IL-2 treatments.

FIG. 10 is a schematic showing, without wishing to be bound by theory, a CD25 and TNFRSF25 two-pathway system of in vivo expansion of Tregs.

### DETAILED DESCRIPTION

In various aspects, the present invention provides for the generation and medical use of Tregs having an activation and functional phenotype associated with effector Treg populations.

In various aspects, the present invention provides for the generation and medical use of Tregs having a markedly greater suppressive activity as shown in vitro and in vivo.

In some aspects, there is provided a method of generating Tregs, in vivo or ex vivo, by contacting a population of Tregs with an agent that targets tumor necrosis factor receptor superfamily member 25 (TNFRSF25), such as a TL1A-Ig fusion protein as described herein (e.g., optionally being 95% similar in amino acid sequence to SEQ ID NO: 1 and SEQ ID NO: 2 or 95% similar in amino acid sequence to SEQ ID NO: 3). In some embodiments, the population of Tregs is additionally contacted with interleukin, such as, for example, IL-2, which can be a low dose of IL-2.

In some aspects, there is provided a method of treating an autoimmune diseases or disorders and/or GVHD and/or preventing transplant rejections and/or delaying acute rejection of a transplanted allograft by administering either: (i) Tregs which have been generated ex vivo by contacting a Treg population with an agent that targets tumor necrosis factor receptor superfamily member 25 (TNFRSF25), such as a TL1A-Ig fusion protein as described herein (optionally being 95% similar in amino acid sequence to SEQ ID NO: 1 and SEQ ID NO: 2 or 95% similar in amino acid sequence to SEQ ID NO: 3), and optionally interleukin (e.g., low dose IL-2), or (ii) an agent that targets TNFRSF25, such as a TL1A-Ig fusion protein as described herein, and optionally interleukin (e.g., low dose IL-2), such agent and interleukin modulating Tregs in vivo.

In various embodiments, the present invention relates to an agonist of TNFRSF25 (also known as DR3), such as the TL1A fusion proteins described herein. TL1A is a type II transmembrane protein belonging to the TNF superfamily and has been designated TNF superfamily member 15 (TNFSF15). TL1A is the natural ligand for TNFRSF25. See U.S. Pat. No. 6,713,061, and Borysenko, et al., Biochem Biophys Res Commun. 2005 Mar. 18; 328(3):794-9, Sheikh, et al., Curr. Cancer Drug Targets. 2004 February; 4(1):97-104, and U.S. publication number 2007/0128184. Human TL1A nucleic acid and amino acid sequences are known and have been described. See, for example GenBank Accession No. CCDS6809.1 (nucleic acid sequence); and GenBank Accession No. EAW87431 (amino acid sequence). Other nucleic acid and amino acid sequences for human TL1A have been described, including, but not limited to GenBank Accession Nos. NM_001204344.1/NP_001191273.1, NM_005118.3/NP_005109.2, NM_001039664.1/NP 001034753.1, NM_148970.1/NP_683871.1, NM 148967.1/NP_683868.1, NM_148966.1/NP_683867.1, NM_148965.1/NP_683866.1, and NM_003790.2/NP_003781.1, each of which is incorporated by reference (including the referenced sequences).

Encompassed herein are non-naturally occurring polynucleotides encoding fusion proteins that specifically bind to TNFRSF25. For example, provided herein are isolated or recombinant nucleic acids containing a polynucleotide sequence which encodes a fusion protein, the fusion protein containing (a) a first polypeptide containing a polypeptide sequence that specifically binds to TNFRSF25, and (b) a second polypeptide containing an Ig polypeptide, or a complementary polynucleotide sequence thereof. A fusion protein described herein can also contain a TL1A polypeptide linked to another second polypeptide that promotes multimerization, e.g., to form a dimer, a trimer, a dimer of trimers, etc. For example, the second polypeptide can be a surfactant protein.

In general, the fusion proteins are agonists of TNFRSF25. In some embodiments, the fusion protein comprises a TL1A polypeptide (a “TL1A fusion protein”). Typically, a TL1A fusion protein encompassed herein induces a signaling response that is similar to the response induced by the natural ligand, TL1A. For example, in some embodiments, TL1A fusion proteins encompassed herein induce proliferation of Treg cells in vitro and/or in vivo. In some embodiments, the TL1A fusion proteins encompassed herein have a T effector cells costimulation effect. Suitable assays for measuring T cell proliferation in vitro and in vivo are known in the art and described in Example 1 (materials and methods). The activity of the TL1A fusion proteins can be measured as described in detail in Khan et al. J. Immunol. 2013 Feb. 15; 190(4):1540-50. In some embodiments, the TL1A fusion protein comprises: a first polypeptide that is capable of binding to TNFRSF25; and at least a second polypeptide. In some embodiments the polypeptide comprises or consists of the extracellular domain of TL1A (e.g., human TL1A extracellular domain) or a fragment thereof that is capable of binding to TNFRSF25 (i.e., a “functionally active fragment”). In some embodiments, the polypeptide is a variant or ortholog of human TL1A or a functionally active fragment thereof. In some embodiments, the human TL1A polypeptide comprises or consists of amino acid residues 68-252 from the TL1A extracellular domain.

In some embodiments, the second polypeptide can be an Ig molecule. For example, the immunoglobulin molecule can be the constant region of an antibody (e.g., IgG, IgA, IgM or IgD). The Ig heavy chains can be divided into three functional regions: Fd (containing VH and CH1 domains), hinge, and Fc. Fd in combination with the light chain forms the “Fab” portion of an antibody. The hinge region is found in IgG, IgA, and IgD classes, and acts as a flexible spacer, allowing the Fab portion to move freely in space. The hinge domains are structurally diverse, varying in both sequence and length among immunoglobulin classes and subclasses. Three human IgG subclasses, IgG1, IgG2, and IgG4, have hinge regions of 12-15 amino acids while IgG3 has approximately 62 amino acids, including 21 proline residues and 11 cysteine residues. The structure of the hinge region is described in detail in Shin et al., Immunological Reviews 130:87 (1992) and in U.S. Patent Application Publication No. 2013/0142793.

For an immunoglobulin fusion protein which is intended for use in humans, the constant regions may be of human sequence origin in order to minimize a potential anti-human immune response. The constant region may also be of human sequence origin in order to provide appropriate effector functions. In some embodiments, the constant region may facilitate multimerization of the fusion protein. Manipulation of sequences encoding antibody constant regions is described in the PCT publication of Morrison and Oi, WO 89/007142. For example, the CH1 domain can be deleted and the carboxyl end of the binding domain is joined to the amino terminus of CH2 through the hinge region. In some embodiments, the Ig molecule comprises a CH2 domain and/or a CH3 domain and/or a hinge region of an immunoglobulin. In some embodiments the second polypeptide is an Ig molecule comprising a hinge region, a CH2 domain and a CH3 domain of an IgG molecule (e.g., human IgG). In some embodiments, the second polypeptide is an Ig molecule comprising a CH2 domain and a CH3 domain of an IgG molecule (e.g., human IgG). In some embodiments, the second polypeptide is an Ig molecule comprising a hinge region and one or more of: a CH2 domain and a CH3 domain of an IgG molecule (e.g., human IgG). In some embodiments, the second polypeptide is an Ig molecule comprising a CH2 domain and at least one of: a hinge region and a CH3 domain of an IgG molecule (e.g., human IgG). In some embodiments, the human immunoglobulin hinge region is an IgG1 hinge region comprising 0, 1, 2, 3, or more cysteine residues.

Fusion proteins encompassed herein can also contain other polypeptides instead of or in addition to the Ig molecules described above. For example, a fusion protein can contain a polypeptide that binds to TNFRSF25 and a surfactant protein, or other polypeptide that facilitates multimerization of the fusion protein.

The nucleic acids disclosed herein, also referred to herein as polynucleotides, may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The sense and anti-sense strands are “complementary” to each other. The nucleic acids which encode TL1A fusion proteins for use according to the compositions and methods disclosed herein may include, but are not limited to: only the coding sequence for the TL1A fusion protein; the coding sequence for the TL1A fusion protein and additional coding sequence; the coding sequence for the TL1A fusion protein (and optionally additional coding sequence) and non-coding sequence, such as introns or non-coding sequences 5′ and/or 3′ of the coding sequence for the TL1A fusion polypeptide, which for example may further include but need not be limited to one or more regulatory nucleic acid sequences that may be a regulated or regulatable promoter, enhancer, other transcription regulatory sequence, repressor binding sequence, translation regulatory sequence or any other regulatory nucleic acid sequence. Thus, as defined above, the term “nucleic acid encoding” or “polynucleotide encoding” a TL1A fusion protein encompasses a nucleic acid which includes only coding sequence for a TL1A fusion polypeptide as well as a nucleic acid which includes additional coding and/or non-coding sequence(s).

Exemplary fusion proteins are described below. It is to be understood that the sequences described below are not limiting. As discussed in more detail below, other TL1A fusion proteins (e.g., those containing fragments, variants, and orthologs of TL1A are also encompassed by the present disclosure, as well as various second polypeptides and/or other functional domains. For example, also encompassed herein are fusion proteins that comprise TL1A and a surfactant protein. In some embodiments, the human TL1A polypeptide comprises or consists of amino acid residues 68-252 from the TL1A extracellular domain.

By way of non-limiting example, in some embodiments the amino acid sequence of the TL1A portion of the human TL1A-Ig fusion protein is:

(SEQ ID NO: 1)
Arg Ala Gln Gly Glu Ala Cys Val Gln Phe Gln Ala
Leu Lys Gly Gln Glu Phe Ala Pro Ser His Gln Gln
Val Tyr Ala Pro Leu Arg Ala Asp Gly Asp Lys Pro
Arg Ala His Leu Thr Val Val Arg Gln Thr Pro Thr
Gln His Phe Lys Asn Gln Phe Pro Ala Leu His Trp
Glu His Glu Leu Gly Leu Ala Phe Thr Lys Asn Arg
Met Asn Tyr Thr Asn Lys Phe Leu Leu Ile Pro Glu
Ser Gly Asp Tyr Phe Ile Tyr Ser Gln Val Thr Phe
Arg Gly Met Thr Ser Glu Cys Ser Glu Ile Arg Gln
Ala Gly Arg Pro Asn Lys Pro Asp Ser Ile Thr Val
Val Ile Thr Lys Val Thr Asp Ser Tyr Pro Glu Pro
Thr Gln Leu Leu Met Gly Thr Lys Ser Val Cys Glu
Val Gly Ser Asn Trp Phe Gln Pro Ile Tyr Leu Gly
Ala Met Phe Ser Leu Gln Glu Gly Asp Lys Leu Met
Val Asn Val Ser Asp Ile Ser Leu Val Asp Tyr Thr
Lys Glu Asp Lys Thr Phe Phe Gly Ala Phe Leu Leu.

In some embodiments, amino acid sequence of the human IgG1 molecule (IgG1 hinge-CH2-CH3 sequence) is:

(SEQ ID NO: 2)
Cys Asp Lys Thr His Thr Cys Pro Pro Cys Pro Ala
Pro Glu Leu Leu Gly Gly Pro Ser Val Phe Leu Phe
Pro Pro Lys Pro Lys Asp Thr Leu Met Ile Ser Arg
Thr Pro Glu Val Thr Cys Val Val Val Asp Val Ser
His Glu Asp Pro Glu Val Lys Phe Asn Trp Tyr Val
Asp Gly Val Glu Val His Asn Ala Lys Thr Lys Pro
Arg Glu Glu Gln Tyr Asn Ser Thr Tyr Arg Val Val
Ser Val Leu Thr Val Leu His Gln Asp Trp Leu Asn
Gly Lys Glu Tyr Lys Cys Lys Val Ser Asn Lys Ala
Leu Pro Ala Pro Ile Glu Lys Thr Ile Ser Lys Ala
Lys Gly Gln Pro Arg Glu Pro Gln Val Tyr Thr Leu
Pro Pro Ser Arg Asp Glu Leu Thr Lys Asn Gln Val
Ser Leu Thr Cys Leu Val Lys Gly Phe Tyr Pro Ser
Asp Ile Ala Val Glu Trp Glu Ser Asn Gly Gln Pro
Glu Asn Asn Tyr Lys Thr Thr Pro Pro Val Leu Asp
Ser Asp Gly Ser Phe Phe Leu Tyr Ser Lys Leu Thr
Val Asp Lys Ser Arg Trp Gln Gln Gly Asn Val Phe
Ser Cys Ser Val Met His Glu Ala Leu His Asn His
Tyr Thr Gln Lys Ser Leu Ser Leu Ser Pro Gly Lys.

An illustrative fusion protein sequence is SEQ ID NO: 3, which shows a restriction enzyme cloning site (residues 229-230 of SEQ ID NO: 3), the residues occurring before the restriction enzyme cloning site correspond to the human IgG1 hinge-CH2-CH3 sequence (residues 1-228 of SEQ ID NO: 3), and the residues following the restriction enzyme cloning site correspond to the human TL extracellular domain sequence (residues 231-422 of SEQ ID NO: 3).

(SEQ ID NO: 3)
Cys Asp Lys Thr His Thr Cys Pro Pro Cys Pro Ala
Pro Glu Leu Leu Gly Gly Pro Ser Val Phe Leu Phe
Pro Pro Lys Pro Lys Asp Thr Leu Met Ile Ser Arg
Thr Pro Glu Val Thr Cys Val Val Val Asp Val Ser
His Glu Asp Pro Glu Val Lys Phe Asn Trp Tyr Val
Asp Gly Val Glu Val His Asn Ala Lys Thr Lys Pro
Arg Glu Glu Gln Tyr Asn Ser Thr Tyr Arg Val Val
Ser Val Leu Thr Val Leu His Gln Asp Trp Leu Asn
Gly Lys Glu Tyr Lys Cys Lys Val Ser Asn Lys Ala
Leu Pro Ala Pro Ile Glu Lys Thr Ile Ser Lys Ala
Lys Gly Gln Pro Arg Glu Pro Gln Val Tyr Thr Leu
Pro Pro Ser Arg Asp Glu Leu Thr Lys Asn Gln Val
Ser Leu Thr Cys Leu Val Lys Gly Phe Tyr Pro Ser
Asp Ile Ala Val Glu Trp Glu Ser Asn Gly Gln Pro
Glu Asn Asn Tyr Lys Thr Thr Pro Pro Val Leu Asp
Ser Asp Gly Ser Phe Phe Leu Tyr Ser Lys Leu Thr
Val Asp Lys Ser Arg Trp Gln Gln Gly Asn Val Phe
Ser Cys Ser Val Met His Glu Ala Leu His Asn His
Tyr Thr Gln Lys Ser Leu Ser Leu Ser Pro Gly Lys
Glu Phe Arg Ala Gln Gly Glu Ala Cys Val Gln Phe
Gln Ala Leu Lys Gly Gln Glu Phe Ala Pro Ser His
Gln Gln Val Tyr Ala Pro Leu Arg Ala Asp Gly Asp
Lys Pro Arg Ala His Leu Thr Val Val Arg Gln Thr
Pro Thr Gln His Phe Lys Asn Gln Phe Pro Ala Leu
His Trp Glu His Glu Leu Gly Leu Ala Phe Thr Lys
Asn Arg Met Asn Tyr Thr Asn Lys Phe Leu Leu Ile
Pro Glu Ser Gly Asp Tyr Phe Ile Tyr Ser Gln Val
Thr Phe Arg Gly Met Thr Ser Glu Cys Ser Glu Ile
Arg Gln Ala Gly Arg Pro Asn Lys Pro Asp Ser Ile
Thr Val Val Ile Thr Lys Val Thr Asp Ser Tyr Pro
Glu Pro Thr Gln Leu Leu Met Gly Thr Lys Ser Val
Cys Glu Val Gly Ser Asn Trp Phe Gln Pro Ile Tyr
Leu Gly Ala Met Phe Ser Leu Gln Glu Gly Asp Lys
Leu Met Val Asn Val Ser Asp Ile Ser Leu Val Asp
Tyr Thr Lys Glu Asp Lys Thr Phe Phe Gly Ala Phe
Leu Leu.

In some embodiments, the amino acid sequence of the human IgG2 molecule (IgG2 hinge-CH2-CH3 sequence) is:

(SEQ ID NO: 4)
Glu Arg Lys Cys Cys Val Glu Cys Pro Pro Cys Pro
Ala Pro Pro Val Ala Gly Pro Ser Val Phe Leu Phe
Pro Pro Lys Pro Lys Asp Thr Leu Met Ile Ser Arg
Thr Pro Glu Val Thr Cys Val Val Val Asp Val Ser
His Glu Asp Pro Glu Val Gln Phe Asn Trp Tyr Val
Asp Gly Val Glu Val His Asn Ala Lys Thr Lys Pro
Arg Glu Glu Gln Phe Asn Ser Thr Phe Arg Val Val
Ser Val Leu Thr Val Val His Gln Asp Trp Leu Asn
Gly Lys Glu Tyr Lys Cys Lys Val Ser Asn Lys Gly
Leu Pro Ala Pro Ile Glu Lys Thr Ile Ser Lys Thr
Lys Gly Gln Pro Arg Glu Pro Gln Val Tyr Thr Leu
Pro Pro Ser Arg Glu Glu Met Thr Lys Asn Gln Val
Ser Leu Thr Cys Leu Val Lys Gly Phe Tyr Pro Ser
Asp Ile Ser Val Glu Trp Glu Ser Asn Gly Gln Pro
Glu Asn Asn Tyr Lys Thr Thr Pro Pro Met Leu Asp
Ser Asp Gly Ser Phe Phe Leu Tyr Ser Lys Leu Thr
Val Asp Lys Ser Arg Trp Gln Gln Gly Asn Val Phe
Ser Cys Ser Val Met His Glu Ala Leu His Asn His
Tyr Thr Gln Lys Ser Leu Ser Leu Ser Pro Gly Lys.

In addition to the amino acid sequences described above, amino acid sequences having certain percent sequence identities to any of the aforementioned sequences are also encompassed. For example, sequences encompassed herein can have about, e.g., 80%, 85%, 90%, 91%, 92%, 93%, 94%, 95%, 96%, 97%, 98%, 99%, or 100% sequence identity to any of SEQ ID NOS: 1, 2, 3, or 4.

Also provided herein are novel methods that include the use of other TNFRSF25 agonists which are known in the art. For example, non-limiting examples of TNFRSF25 agonists that may be used in the methods disclosed herein include, e.g., small molecules, antibodies, and fusion proteins. Non-limiting examples of such TNFRSF25 agonists are described, e.g., in U.S. pre-grant publication Nos. 2011/0243951, 2012/0029472, and 2012/0135011, all by Podack et al. Methods for preparing anti-TNFRSF25 antibodies are described in U.S. 2012/0029472 by Podack et al. The present methods envision the use of any suitable TNFRSF25 agonists known in the art. In some embodiments, TNFRSF25 agonists are ones which enhance the expansion of Treg cells.

In some embodiments, a TNFRSF25 agonist is a small molecule. Chemical agents, referred to in the art as “small molecules” are typically organic, non-peptide molecules, having a molecular weight less than 10,000 Da, preferably less than 5,000 Da, more preferably less than 1,000 Da, and most preferably less than 500 Da. This class of modulators includes chemically synthesized molecules, for instance, compounds from combinatorial chemical libraries. Synthetic compounds may be rationally designed or identified by screening compound libraries for TNFRSF25-modulating activity according to methods known in the art. Alternative appropriate modulators of this class are natural products, particularly secondary metabolites from organisms such as plants or fungi, which can also be identified by screening compound libraries for TNFRSF25-modulating activity. Methods for generating and obtaining small molecules are well known in the art (see, e.g., Schreiber, Science 2000; 151:1964-1969; Radmann et al., Science 2000; 151:1947-1948).

In some embodiments, the present disclosure provides nucleic acids encoding TL1A fusion proteins and compositions that contain the TL1A fusion proteins. Also described herein are methods of producing TL1A fusion proteins. The method can include for example, introducing into a population of cells a nucleic acid encoding the TL1A fusion protein, wherein the nucleic acid is operatively linked to a regulatory sequence effective to produce the fusion protein polypeptide encoded by the nucleic acid; and culturing the cells in a culture medium to produce the polypeptide. In some embodiments, the method can further include isolating the fusion protein polypeptide from the cells or culture medium. The nucleic acid can also further contain a third nucleotide sequence that encodes a secretory or signal peptide operably linked to the fusion protein. In some embodiments, the fusion protein is secreted from the host cell as a fusion protein homomultimer (e.g., as a dimer of trimers). In some embodiments, the fusion protein homomultimer is recovered from the culture medium, the host cell or host cell periplasm. Further, the fusion protein homomultimer can contain one or more covalent disulfide bonds between a cysteine residue of the first fusion protein and at least one cysteine residue of one or more additional fusion proteins.

In any of the compositions and methods disclosed herein comprising an interleukin, the interleukin can be any interleukin that achieves the desired synergistic effect on the expansion of Treg cells, e.g., when administered in a combination therapy with an agonist of TNFRSF25. In some embodiments, the interleukin is IL-2. In some embodiments, the interleukin is IL-7. In some embodiments, the interleukin is IL-15.

Also encompassed herein are analogs of IL-2, e.g., agonist and partial agonist IL-2 analogs (e.g., IL-2 muteins). Such analogs are known in the art. A non-limiting example of an agonist IL-2 analog includes, e.g., BAY 50-4798 (see Margolin et al. Clin Cancer Res Jun. 1, 2007 13; 3312; for other examples, see also, Imler and Zurawski. J Biol. Chem. 1992 Jul. 5; 267(19):13185-90. Furthermore, in vitro screening assays for determining whether a compound is an IL-2 analog (i.e., maintains the ability to bind to the high affinity IL-2 receptor and initiate T cell proliferation) are known in the art. See, e.g., Zurawski and Zurawski. EMBO J. 1992 November; 11(11): 3905-3910; “The Interleukin 2 Receptor” Annual Review of Cell Biology; Vol. 5: 397-425 (Volume publication date November 1989; and “The Biology of Interleukin-2”; Annual Review of Immunology; Vol. 26: 453-479 (Volume publication date April 2008).

In some embodiments, provided herein is a method of treating or preventing GVHD in a transplant recipient, comprising administering a human TL1A-Ig fusion protein to a transplant donor, wherein the fusion protein comprises (a) a first polypeptide comprising an extracellular domain of a human TL1A polypeptide or a fragment thereof that specifically binds to TNFRSF25, and (b) a second polypeptide comprising an immunoglobulin (Ig) polypeptide. The administration can occur at least 3 times, and the transplant comprises Tregs from the transplant donor. In some embodiments, the administration of the TL1A-Ig fusion protein to the transplant donor can occur prior to transplant.

In some embodiments, the graft versus host disease is reduced. The transplant can comprise donor hematopoietic cells, donor stem cells, or donor bone marrow cells. In some embodiments, the graft versus host disease is acute graft-versus-host-disease (aGVHD) or chronic graft-versus-host-disease (cGVHD).

In embodiments, the present methods relate to treating or preventing GVHD in a transplant recipient. In embodiments, the transplant recipient is a cancer patient, e.g. one who has received or is receiving radiation or chemotherapy and consequently has received or is an allogeneic hematopoietic stem cell transplant (allo-HSCT). In embodiments, the transplant recipient has blood or bone marrow cancer. In embodiments, the transplant recipient is the recipient of a hematopoietic stem cell transplant. In certain embodiments, the cancer is selected from leukemia, lymphoma, myeloma, and myelodysplasia. In certain embodiments, the cancer is selected from osteosarcoma, Ewing tumors, chordomas, and chondrosarcomas.

GVHD is the deterioration of cells or tissues that are transplanted from a donor to a recipient due to the recognition by the immune system of the recipient that the cells or tissues are foreign. Thus, because Class I MHC are on more cells of the body, it is most desirable to transplant cells and tissues from people that have highest matching Class I MHC profiles followed by the highest matching Class II MHC profiles. Thus, in most transplant recipients, GVHD is due to activation of the immune system to mismatched Class II MHC molecules and other polymorphic proteins (minor histocompatibility antigens).

One option for treating cancers of the blood and bone marrow is to kill existing blood and marrow cells, e.g., through radiation or chemotherapy, and transplant similar cells from a healthy donor, referred to as an allogeneic hematopoietic stem cell transplant (allo-HSCT). Chemotherapy for bone marrow remission typically includes prednisone, vincristine, and an anthracycline drug; other drug plans may include L-asparaginase or cyclophosphamide. Another option is prednisone, L-asparaginase, and vincristine. Another options included methotrexate and 6-mercaptopurine (6-MP). In embodiments, the transplant recipient is receiving treatment or has received treatment with any of these agents.

In embodiments, the present methods relate to acute and chronic forms of GVHD. The acute or fulminant form of the disease (aGVHD) is normally observed within the first 100 days post-transplant, and is a major challenge to the effectiveness of transplants owing to the associated morbidity and mortality. The chronic form of graft-versus-host-disease (cGVHD) normally occurs after 100 days. The appearance of moderate to severe cases of cGVHD adversely influences long-term survival. After bone marrow transplantation, T cells present in the graft, either as contaminants or intentionally introduced into the host, attack the tissues of the transplant recipient after perceiving host tissues as antigenically foreign. The T cells produce an excess of cytokines, including TNF alpha and interferon-gamma (IFNγ). A wide range of host antigens can initiate graft-versus-host-disease, among them the human leukocyte antigens (HLAs). However, graft-versus-host disease can occur even when HLA-identical siblings are the donors. Classically, acute graft-versus-host-disease is characterized by selective damage to the liver, skin and mucosa, and the gastrointestinal tract. Additional studies show that that other graft-versus-host-disease target organs include the immune system (such as the bone marrow and the thymus) itself, and the lungs in the form of idiopathic pneumonitis. Chronic graft-versus-host-disease also attacks the above organs, but over its long-term course can also cause damage to the connective tissue and exocrine glands.

In embodiments, the present methods relate to treating or preventing acute GVHD. In embodiments, the present methods treat a patient who has one or more risk factors of acute GVHD, such as HLA “mismatch,” or unrelated donor, older patient age, female donor to male recipient, intensity of the conditioning regimen or total body irradiation during conditioning regimen, and donor lymphocyte infusion. In embodiments, the present methods treat or prevent symptoms of acute GVHD, such as skin rash, gastrointestinal (GI) tract disorders, and liver symptoms.

In embodiments, the present methods relate to treating or preventing chronic GVHD. In embodiments, the present methods treat a patient who has one or more risk factors of chronic GVHD, such as HLA mismatch or unrelated donor, older patient age, older donor age, female donor for male recipient and number of children the female donor has had, stem cell source, stem cells retrieved from peripheral blood have a higher risk of causing chronic GVHD than stem cells retrieved from bone marrow, stem cells retrieved from cord blood have the lowest risk of causing chronic GVHD, and prior acute GVHD. In embodiments, the present methods treat or prevent symptoms of chronic GVHD, such as symptoms of the eyes, mouth, skin, nails, scalp and body hair, gastrointestinal (GI) tract, lungs, liver, muscles and joints, and genitals and sex organs.

In embodiments, the present methods relate to GVHD as defined by one of more of the Billingham Criteria: 1) administration of an immunocompetent graft, with viable and functional immune cells; 2) the recipient is immunologically histoincompatible; and 3) the recipient is immunocompromised and therefore cannot destroy or inactivate the transplanted cells.

In embodiments, the present methods relate to treating or preventing GVHD in a patient/transplant recipient who is undergoing a GVHD treatment. In embodiments, the present methods relate to treating or preventing GVHD in using the present fusion proteins in combination therapies with a GVHD treatment. In embodiments, the present fusion proteins reduce or ameliorate one or more side effects of a GVHD treatment. Illustrative GVHD treatments are immunosuppression agents, such as corticosteroids (such as methylprednisolone or prednisone) and other immunosuppressive drugs. An illustrative GVHD treatment, in some embodiments, is prednisone. Other illustrative GVHD treatments include ibrutinib (e.g. IMBRUVICA), mycophenolate mofetil, mTOR inhibitors, such as sirolimus (rapamycin), everolimus, calcineurin inhibitors, such as tacrolimus or cyclosporine, ciclosporin, monoclonal antibodies such as infliximab (e.g. REMICADE), tocilizumab (e.g. ACTEMRA), alemtuzumab (e.g. CAMPATH), basiliximab (e.g. SIMULECT), daclizumab (e.g. ZINBRYTA), and denileukin diftitox (e.g. ONTAK), antithymocyte globulin (ATG), anti-lymphocyte globulin (ALG), pentostatin (e.g. NIPENT), ruxolitinib (e.g. JAKAFI), and photopheresis.

In embodiments, the present methods pertain to patients who fail to respond to steroid therapy are labeled “steroid-refractory”. In embodiments, the present methods pertain to patients who fail one or more lines of systemic GVHD therapy.

In some embodiments, the administration of the TL1A-Ig fusion protein is also to the transplant recipient, and the administration to the transplant recipient can occur after the transplant. In some embodiments, the administration is to both the transplant donor and transplant recipient. In some embodiments, the fusion protein causes a sustained increase in Treg cells in the transplant donor and/or transplant recipient. In some embodiments, the fusion protein does not cause substantial Treg suppression in the transplant donor and/or transplant recipient. In some embodiments, the fusion protein does not cause substantial Treg anergy in the transplant donor and/or transplant recipient. In some embodiments, the administration of the fusion protein is to one or both of the transplant donor and the transplant recipient occurs.

In some aspects, provided herein is a method of reducing or preventing an immune response in a solid organ transplant recipient, comprising administering a human TL1A-Ig fusion protein to the solid organ transplant recipient, wherein the fusion protein comprises (a) a first polypeptide comprising an extracellular domain of a human TL1A polypeptide or a fragment thereof that specifically binds to TNFRSF25, and (b) a second polypeptide comprising an immunoglobulin (Ig) polypeptide. In the method, the administration can occur at least 3 times, and the immune response is a rejection of the solid organ transplant.

In some embodiments, the method of reducing or preventing an immune response in a solid organ transplant recipient prevents a solid organ transplant rejection. In some embodiments, the method reduces the likelihood of solid organ transplant rejection. The fusion protein can cause a sustained increase in Treg cells in the solid organ transplant recipient. The sustained increase in Treg cells can comprise a substantially similar level of Treg cells in the solid organ transplant recipient after the first and last fusion protein administrations. For example, if the administration of the fusion protein occurs 3 times, the increase in Treg cells remains at a substantially similar level in the solid organ transplant recipient after the first and third fusion protein administrations.

In some embodiments, the fusion protein does not cause substantial Treg suppression in the transplant donor and/or transplant recipient. In some embodiments, the fusion protein does not cause substantial Treg anergy in the transplant donor and/or transplant recipient.

In some embodiments, the solid organ is selected from lung, kidney, heart, liver, pancreas, thymus, gastrointestinal tract, cornea, eye, and composite allografts. Composite allograft transplantation can be, e.g., intestinal/multivisceral transplantation (which may or may not include liver) and its variants.

In embodiments in accordance with the present disclosure, the fusion protein can be administered in various doses. For example, the administration can occur at least 7 times, at least 10 times, at least 14 times, about 3-7 times, about 3-14 times, about 3-21 times, about 3 times, about 7 times, about 10 times, or about 14 times. In some embodiments, the administration occurs daily, which can be, for example, twice daily. In some embodiments, the administration occurs daily for 3-7 days, daily for 7-14 days, daily for 7-21 days, daily for at least 7 days, daily for at least 10 days, or daily for at least 21 days. The administration in any of the above doses can occur before the transplant or concurrently with the transplant. In embodiments in which the transplant is a solid organ transplant, the administration of the fusion protein in any of the above doses can occur after the solid organ transplant, or before and after the solid organ transplant.

In some embodiments, the first polypeptide comprises (a) the amino acid sequence of SEQ ID NO: 1, or (b) an amino acid sequence that has at least 95% sequence identity to SEQ ID NO: 1. The Ig polypeptide can comprise one or more of a hinge region, a CH2 domain, and a CH3 domain of an IgG polypeptide. In some embodiments, the Ig polypeptide comprises one or more of a hinge region, a CH2 domain, and a CH3 domain of a human IgG1. In other embodiments, the Ig polypeptide comprises one or more of a hinge region, a CH2 domain, and a CH3 domain of a human IgG2.

In some embodiments, the Ig polypeptide comprises all of a hinge region, CH2 domain, and CH3 domain of an IgG polypeptide. In some embodiments, the Ig polypeptide comprises all of a hinge region, CH2 domain, and CH3 domain of a human IgG1. In some embodiments, the Ig polypeptide comprises (a) the amino acid sequence of SEQ ID NO: 2, or (b) an amino acid sequence that has at least 95% sequence identity to SEQ ID NO: 2. In some embodiments, the Ig polypeptide comprises all of a hinge region, CH2 domain, and CH3 domain of a human IgG2.

In some embodiments, the Ig polypeptide comprises (a) the amino acid sequence of SEQ ID NO: 4, or (b) an amino acid sequence that has at least 95% sequence identity to SEQ ID NO: 4.

In some embodiments, the fusion protein can comprise (a) the amino acid sequence of SEQ ID NO: 1, or an amino acid sequence that has at least 95% sequence identity to SEQ ID NO: 1, and (b) the amino acid sequence of SEQ ID NO: 2 or SEQ ID NO: 4, or an amino acid sequence that has at least 95% sequence identity to SEQ ID NO: 2 or SEQ ID NO: 4.

In some embodiments, the method of treating or preventing GVHD in a transplant recipient, comprising administering a human TL1A-Ig fusion protein to a transplant donor, further includes administering interleukin-2 (IL-2). In some embodiments, the method of reducing or preventing an immune response in a solid organ transplant recipient, which comprises administering the fusion protein as described herein to the solid organ transplant recipient, also further comprises administering IL-2. In these embodiments, or in any other embodiments in accordance with the present disclosure, IL-2 can be administered as a low dose of IL-2. The low dose of IL-2 can be less than 1 million units per square meter per day. In some embodiments, the low dose of IL-2 is an amount in the range of about 30,000 to about 300,000 units per square meter per day. In some embodiments, the low dose of IL-2 is about 300,000 units per square meter per day. In some embodiments, the low dose of IL-2 is about 30,000 units per square meter per day. The administration of a low dose IL-2, in any of the above doses, can be sequential with the fusion protein or concurrent with the fusion protein.

In some embodiments, a human TL1A-Ig fusion protein administered to a transplant recipient for treating or preventing GVHD in the transplant recipient, or a human TL1A-Ig fusion protein administered to a solid organ transplant recipient for reducing or preventing an immune response in the solid organ transplant recipient can be administered in combination with one or more additional drugs, e.g. anti-rejection drugs that are suitable for prevention, treatment of or reduction of symptoms of GVHD and/or solid organ transplant rejection. Non-limiting examples of anti-rejection drugs that can be administered to a (solid organ) transplant recipient include corticosteroids (e.g., prednisone or methylprednisone); calcineurin inhibitors (e.g., cyclosporine and/or tacrolimus); azathioprine; everolimus; sirolimus; mycophenolate mofetil; monoclonal antibodies such as, e.g., infliximab, basiliximab, tocilizumab, dacilzumab, rituximab, alemtuzumab, and denileukin diftitox; ibrutinib; pentostatin; ruxolitinib; and polyclonal antibodies such as, e.g., antithymocite globulin. The anti-rejection drug, or a combination of the anti-rejection drugs can be administered in the same formulation as the fusion protein or in a separate formulation. Also, the anti-rejection drugs can be administered in the same formulation or in separate formulations. Any one or more agents of the fusion protein in accordance with the present disclosure and the anti-rejection drug(s) can be administered sequentially or simultaneously with the other agent(s).

A combination therapy in which the fusion protein described herein can be administered sequentially or simultaneously with one or more anti-rejection drugs (and in which the anti-rejection drugs can be administered sequentially or simultaneously) can include administration of IL-2 (e.g., a low dose of IL-2), which can also be administered sequentially or simultaneously with the fusion protein and/or with one or more anti-rejection drugs.

In various embodiments, the present invention relates to the generation or modulation of Tregs. A “T regulatory cell” or “Treg cell” refers to a cell that can modulate a T cell response. Treg cells express the transcription factor Foxp3, which is not upregulated upon T cell activation and discriminates Tregs from activated effector cells. Tregs are identified by the cell surface markers CD25, CTLA4, and GITR. Several Treg subsets have been identified that have the ability to inhibit autoimmune and chronic inflammatory responses and to maintain immune tolerance in tumor-bearing hosts. These subsets include interleukin 10- (IL-10-) secreting T regulatory type 1 (Tr1) cells, transforming growth factor-β− (TGF-β−) secreting T helper type 3 (Th3) cells, and “natural” CD4⇄/CD25+ Tregs (Trn) (Fehervari and Sakaguchi. J. Clin. Invest. 2004, 114:1209-1217; Chen et al. Science. 1994, 265: 1237-1240; Groux et al. Nature. 1997, 389: 737-742).

In some embodiments, a method for generating Tregs in vivo is provided that comprises administering to a subject in need thereof a human TL1A-Ig fusion protein to expand and selectively activate a population of Tregs. The fusion protein comprises (a) a first polypeptide comprising an extracellular domain of a human TL1A polypeptide or a fragment thereof that specifically binds to TNFRSF25, and (b) a second polypeptide comprising an immunoglobulin (Ig) polypeptide. In some embodiments, the method further comprises administering a low dose of IL-2.

In some embodiments, a method for generating Tregs ex vivo is provided that comprises (a) isolating a population of Tregs from a subject, and (b) contacting the isolated population of Tregs with a human TL1A-Ig fusion protein to expand and selectively activate a population of Tregs. The fusion protein comprises (a) a first polypeptide comprising an extracellular domain of a human TL1A polypeptide or a fragment thereof that specifically binds to TNFRSF25, and (b) a second polypeptide comprising an immunoglobulin (Ig) polypeptide. In some embodiments, the method further comprises contacting the Tregs with a low dose of IL-2.

In the above methods for generating Tregs, the low dose of IL-2 can be, e.g., less than 1 million units per square meter per day, in the range of about 30,000 to about 300,000 units per square meter per day, about 300,000 units per square meter per day, or about 30,000 units per square meter per day.

As described above, in some embodiments, the methods described herein are useful for treating autoimmune diseases, alloimmune responses, or any other disease, disorder or condition that involves a T cell response (e.g., in a patient in need thereof). Generally, these are conditions in which the immune system of an individual (e.g., activated T cells) attacks the individual's own tissues and cells, or implanted tissues, cells, or molecules (as in a graft or transplant). Non-limiting examples of diseases and disorders that can be treated according to the methods described herein, include, e.g., autoimmune disease or disorder (e.g., IBD and rheumatoid arthritis), transplant rejection, GVHD, inflammation, asthma, allergies, and chronic infection.

In some embodiments, a method for preventing, reducing or ameliorating an autoimmune disease or disorder in a subject in need thereof is provided that includes administering a human TL1A-Ig fusion protein to the subject, the fusion protein comprising (a) a first polypeptide comprising an extracellular domain of a human TL1A polypeptide or a fragment thereof that specifically binds to TNFRSF25, and (b) a second polypeptide comprising an immunoglobulin (Ig) polypeptide. The method can further include administering a low dose of IL-2.

In some embodiments, a method for preventing, reducing or ameliorating an autoimmune disease or disorder in a subject in need thereof is provided, that includes administering a population of Tregs to the subject, wherein the population of Tregs has been treated with a human TL1A-Ig fusion protein. The fusion protein comprises (a) a first polypeptide comprising an extracellular domain of a human TL1A polypeptide or a fragment thereof that specifically binds to TNFRSF25, and (b) a second polypeptide comprising an immunoglobulin (Ig) polypeptide. In some embodiments, the population of Tregs has been further treated with a low dose of IL-2.

In some embodiments, the Tregs are administered at lower doses than a treatment with untreated Tregs (e.g. 30% lower dose, 40% lower dose, 50% lower dose, 60% lower dose, 70% lower dose, 80% lower dose, 90% lower dose, 100% lower dose).

In any of the embodiments described herein, or in a combination of the embodiments, the low dose of IL-2 can be, e.g., less than 1 million units per square meter per day, in the range of about 30,000 to about 300,000 units per square meter per day, about 300,000 units per square meter per day, or about 30,000 units per square meter.

For transplant rejection and GVHD associated disorders, a patient in need of treatment can be a patient who is undergoing or about to undergo induction therapy in preparation for a solid organ or stem cell transplant, a patient who is a solid organ or stem cell transplant recipient and is undergoing or is about to undergo maintenance therapy, a patient who is a solid organ or stem cell transplant recipient (and the therapy, e.g., TL1A fusion protein or combination therapy comprising administration of a TNFRSF25 agonist and an interleukin (e.g., IL-2, IL-7, IL-15, or an analog thereof) is administered in order to facilitate early withdrawal of maintenance immunosuppressive therapy), an allergic patient (and the therapy, e.g., TL1A fusion protein or combination therapy comprising administration of a TNFRSF25 agonist and an interleukin (e.g., IL-2, IL-7, IL-15) is administered to reduce symptoms of a specific allergic reaction); a patient who is receiving or about to receive a vaccine (and the therapy, e.g., TL1A fusion protein or combination therapy comprising administration of a TNFRSF25 agonist and an interleukin (e.g., IL-2, IL-7, IL-15, or an analog thereof) is administered in order to enhance antigen-specific T cell responses stimulated by the vaccine or in order to enhance T cell memory immune responses), or a patient being treated or about to be treated with an immune checkpoint inhibitor (e.g., CTLA-4 or PD-1 inhibitor) (and the therapy, e.g., TL1A fusion protein or combination therapy comprising administration of a TNFRSF25 agonist and an interleukin (e.g., IL-2, IL-7, IL-15, or an analog thereof), is administered in order to enhance T cell immune responses).

Illustrative autoimmune diseases that can be treated with the methods of the present disclosure include, e.g., type I diabetes, multiple sclerosis, thyroiditis (such as Hashimoto's thyroiditis and Ord's thyroiditis), Grave's disease, systemic lupus erythematosus, scleroderma, psoriasis, arthritis, rheumatoid arthritis, alopecia greata, ankylosing spondylitis, autoimmune hemolytic anemia, autoimmune hepatitis, Behcet's disease, Crohn's disease, dermatomyositis, glomerulonephritis, Guillain-Barre syndrome, IBD, lupus nephritis, myasthenia gravis, myocarditis, pemphigus/pemphigoid, pernicious anemia, polyarteritis nodosa, polymyositis, primary biliary cirrhosis, rheumatic fever, sarcoidosis, Sjogren's syndrome, ulcerative colitis, uveitis, vitiligo, and Wegener's granulomatosis.

Illustrative alloimmune responses that can be treated with the methods of the present disclosure include GVHD and transplant rejection. Thus, for example, the fusion proteins disclosed herein can be administered as an “induction therapy” in preparation for a solid organ or stem cell transplant, or as “maintenance therapy” in solid organ or stem cell transplant recipients, and can also be administered to a solid organ or stem cell transplant recipient in order to facilitate early withdrawal of maintenance immunosuppressive therapy.

Any aspect or embodiment described herein can be combined with any other aspect or embodiment as disclosed herein.

The invention will be further described in the following examples, which do not limit the scope of the invention described in the claims.

### EXAMPLES

### Example 1

Phenotypic and Functional Difference Between CD25 and TNFRSF25 “Two Pathway” In Vivo Expanded Tregs and Unexpanded Tregs.

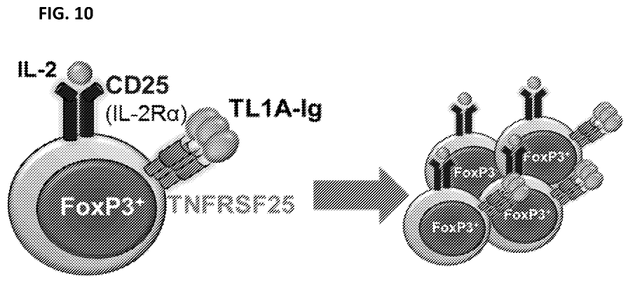

Tregs are non-redundant mediators of immunity and tolerance. Transfer of Tregs is a promising therapy for autoimmune disease, organ rejection and GVHD following allogeneic hematopoietic stem cell transplantation (aHSCT). Cell purity, functional stability and practical issues, including economics, pose challenges to employ ex-vivo Treg expansion. The CD25 and TNFRSF25 “two-pathway” approach described herein (see FIG. 10), markedly expands (>50% of CD4+ T cells) and selectively activates Tregs in vivo by targeting TNFRSF25 (with TL1A-Ig fusion protein) and CD25 (with low dose IL-2).

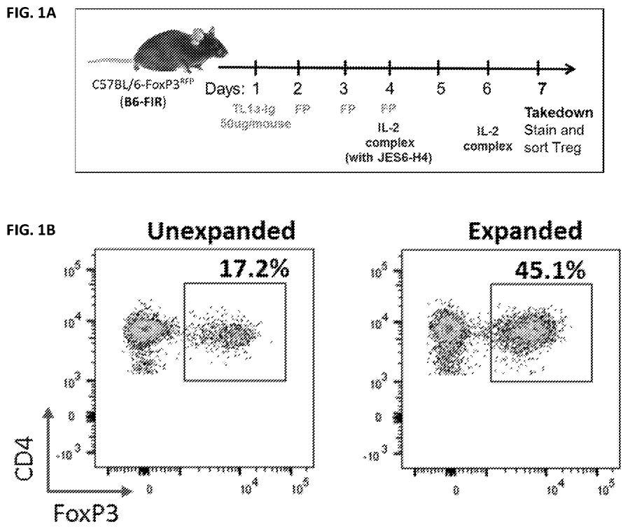

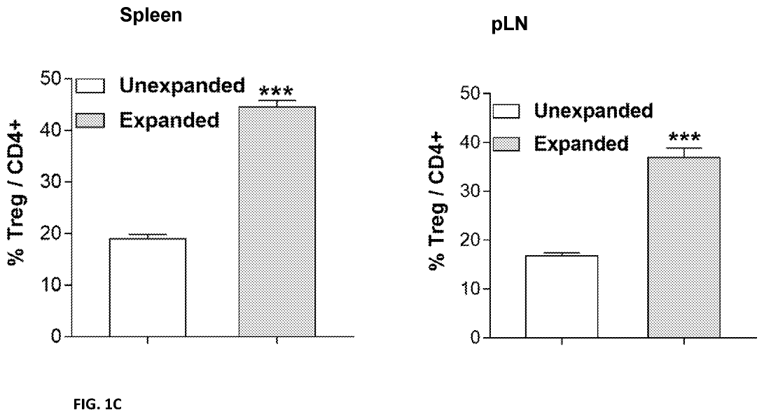

To examine how the CD25 and TNFRSF25, two-pathway Treg expansion affects FoxP3+CD4+ Treg subset distribution, C57BL/6-FoxP3^{RFP}, (B6-FIR) mice were treated with TL1a-Ig, IL-2 complex and IL-2 complex with JES6-H4 for 7 days (see FIG. 1A). Following day 7, the cells were stained and sorted for Tregs. In vivo treatment with TL1A-Ig+low dose IL-2 induced a strong increase in the overall levels of CD4+FoxP3+ within the CD4+ T cell compartment (see FIG. 1B). Results showed that in vivo expansion (via TL1A-Ig+low dose IL-2) induced a strong increase in the percentage of CD4⁺FoxP3⁺ Tregs within the CD4⁺ T cell compartment in the spleen and peripheral lymph nodes (pLN) (see FIG. 1C).

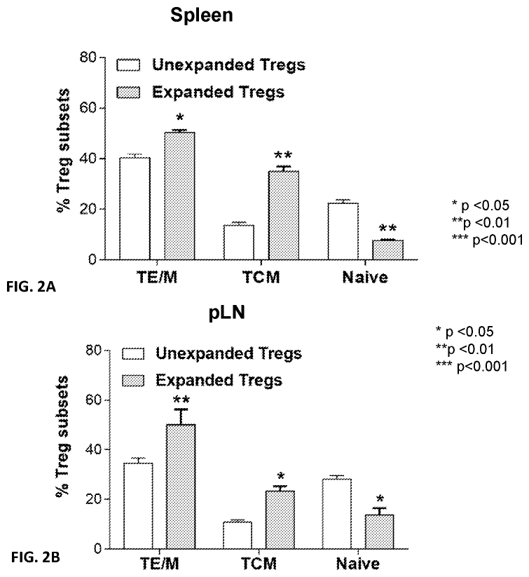

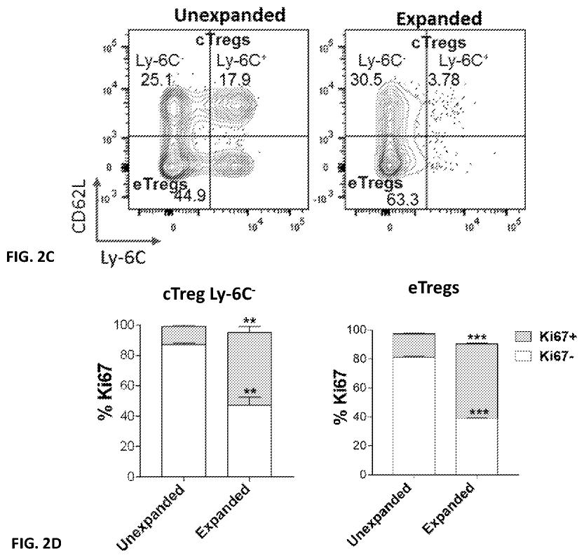

The “two-pathway” expanded Tregs exhibited significantly fewer naive Tregs and increased central memory TCM (CD62-LhiCD44+) and effector/memory TE/M (CD62-LloCD44+) Tregs in the spleen and peripheral lymph nodes (pLN) (see FIGS. 2A and 2B). Furthermore, expanded Tregs exhibited a significant decrease in Ly6C expression (see FIGS. 2C and 2D).

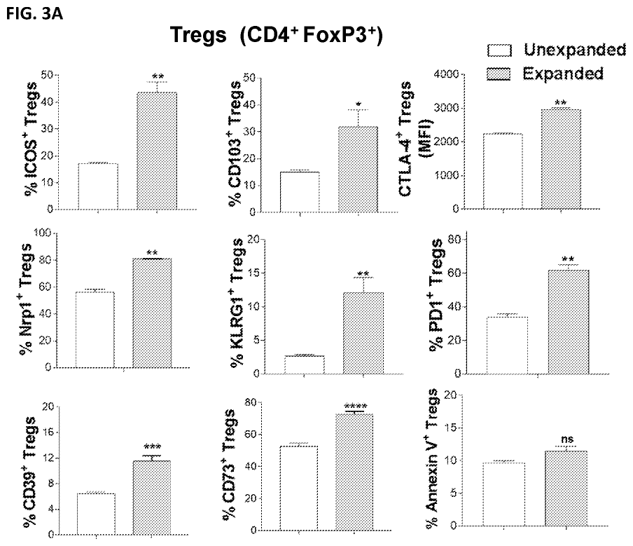

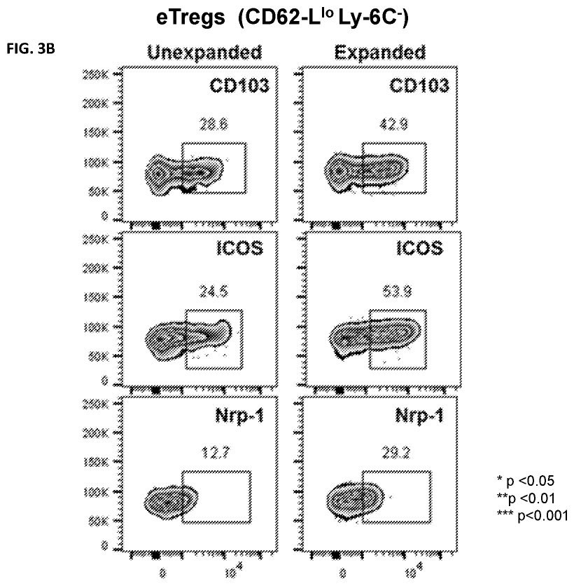

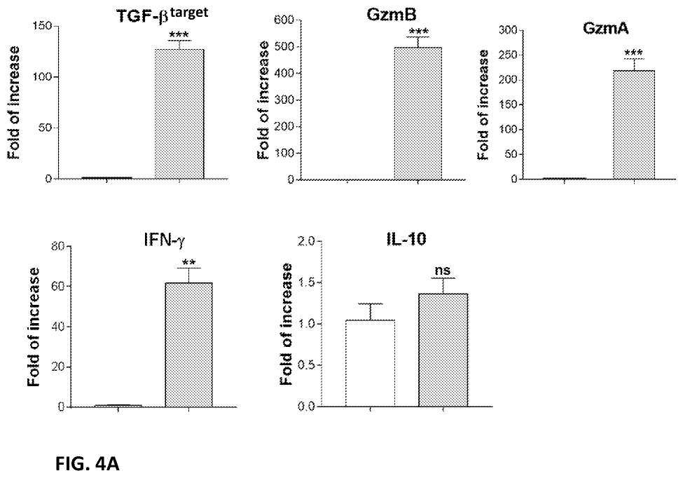

The experiments demonstrated that IL-2 and TL1A-Ig driven in vivo expansion modify Treg effector molecules and function. In FIGS. 3A and 3B, higher levels of activation and functional markers were observed in the peripheral lymph nodes between the expanded vs unexpanded Tregs (similar results were observed in the spleen). Increased expression of these molecules by Tregs are associated with differentiation, co-stimulation, DC engagement and migration.

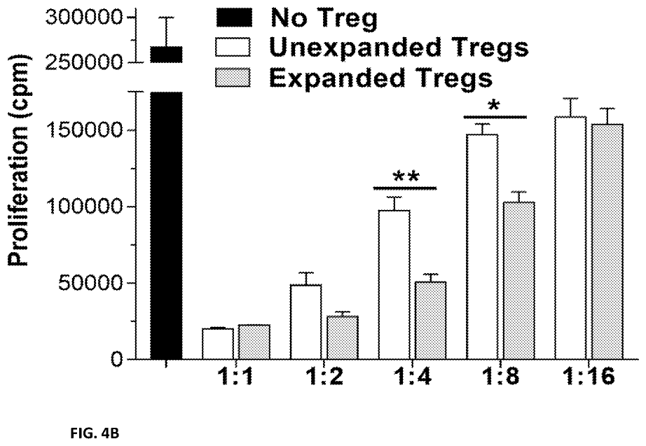

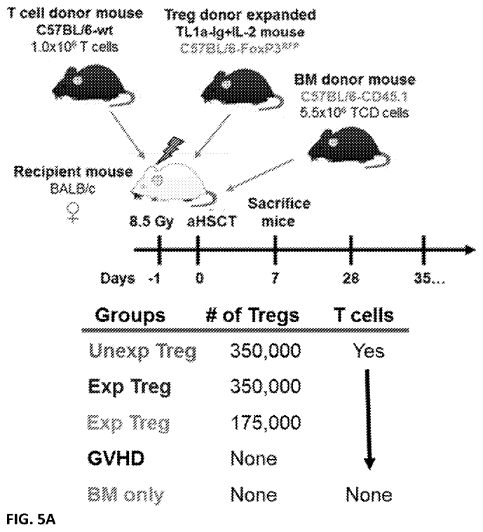

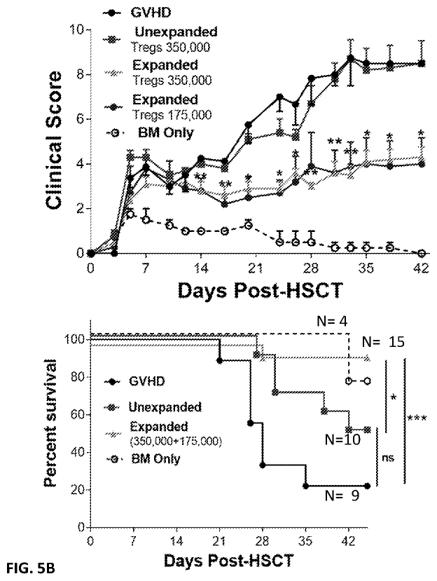

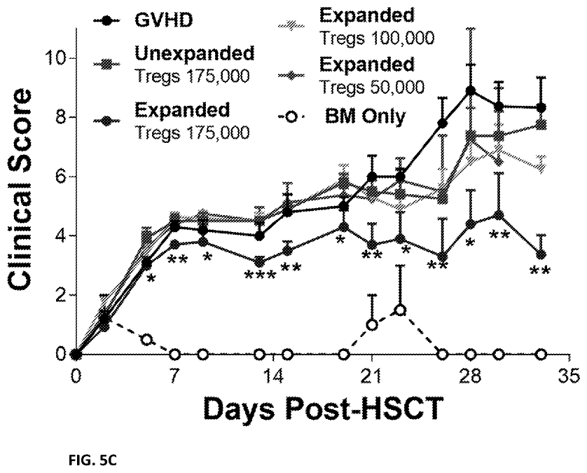

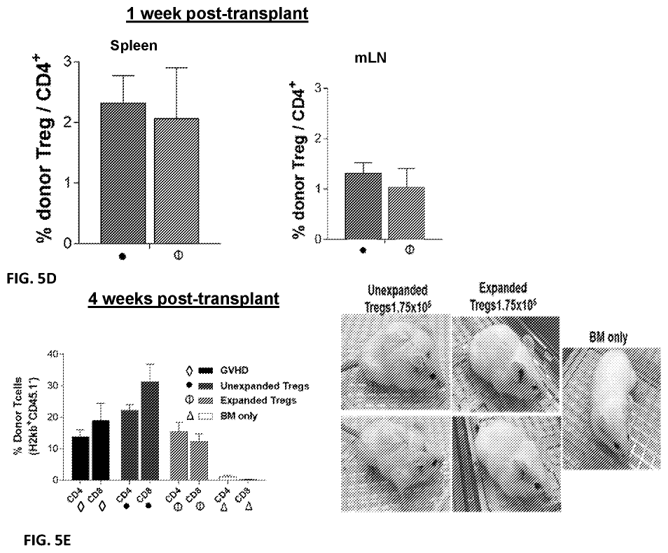

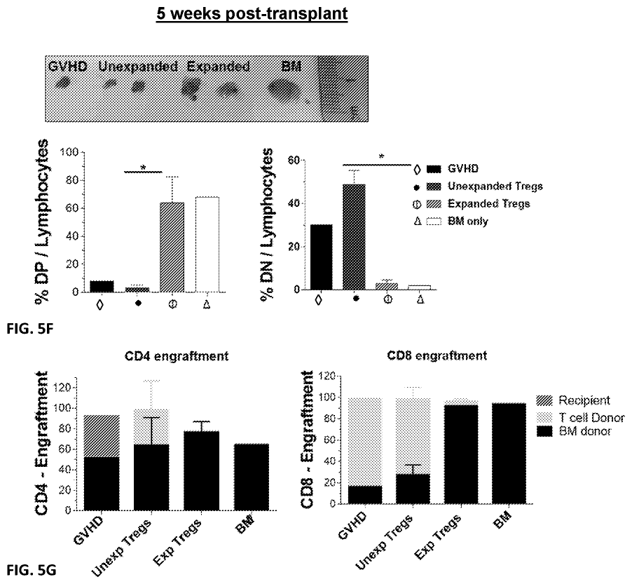

The two-pathway expanded Tregs mediate superior GVHD amelioration when compared to unexpanded Tregs. As shown in FIGS. 4A and 4B, expanded Tregs exhibited higher mRNA levels of Treg effector molecules and mediated enhanced in vitro suppressor activity when compared to unexpanded Tregs. Sorted Tregs from indicated mice were analyzed by real-time PCR and suppressor assays. Further, MHC-mismatched aHSCT (B6-BALB/c mice) was performed using sorted expanded or unexpanded donor Tregs combined with T conventional cells. Using only 3.5×10⁵ unexpanded Tregs+1.0×10⁶ splenic T cells was not sufficient to prevent GVHD. However, transfer of as low as 1.75×10⁵ expanded CD4+FoxP3⁺ cells (ratio 0.2:1 Treg/Teff) was sufficient to diminish GVHD. Lower numbers of expanded Tregs (0.05 to 1.0×10⁵) were not sufficient to prevent GVHD (see FIGS. 5B-5G).

Accordingly, CD25 and TNFRSF25-two-pathway-stimulated Tregs induced an activation and functional phenotype associated with effector Treg populations. These two-pathway expanded Tregs exhibited a markedly greater suppressive activity as shown in vitro and in vivo by amelioration of GVHD following pre-clinical MHC-mismatched aHSCT. Since low numbers of Tregs can diminish GVHD across an MHC-mismatched transplant, this strategy could be considered to treat autoimmune diseases and prevent solid organ rejection.

### Recent Thymic Emigrant (RTE) Analysis

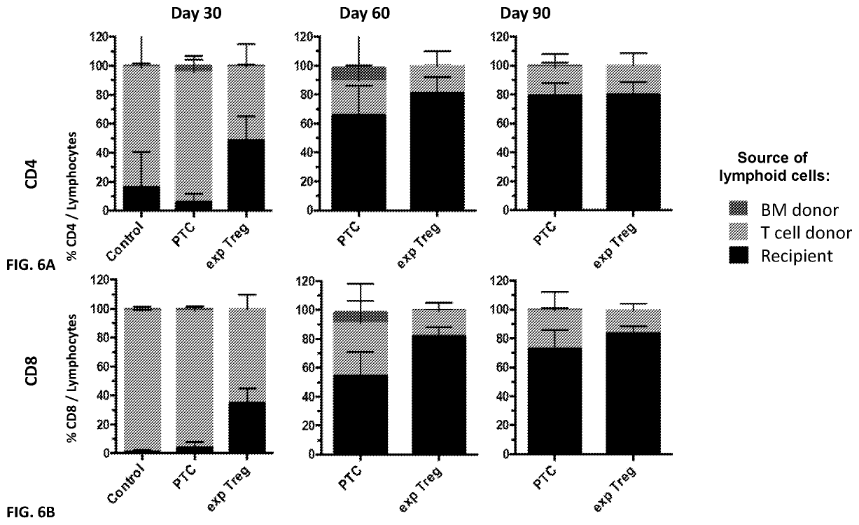

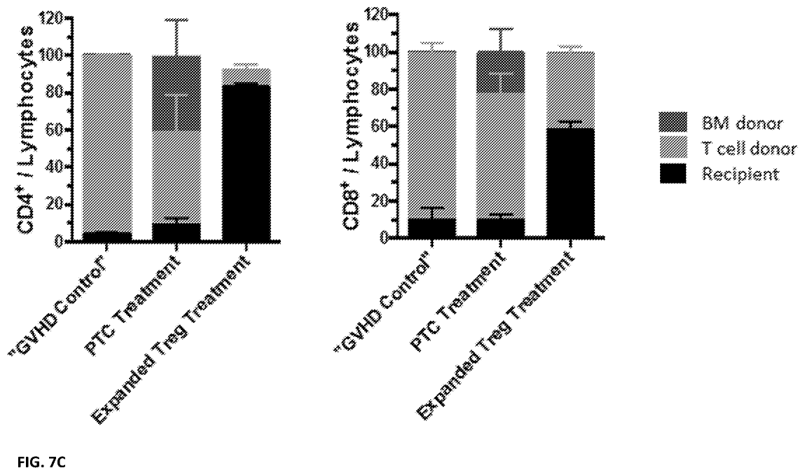

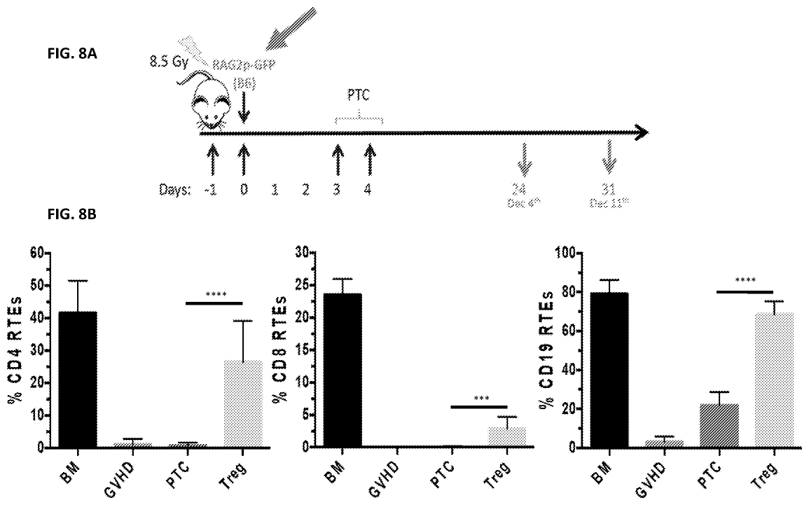

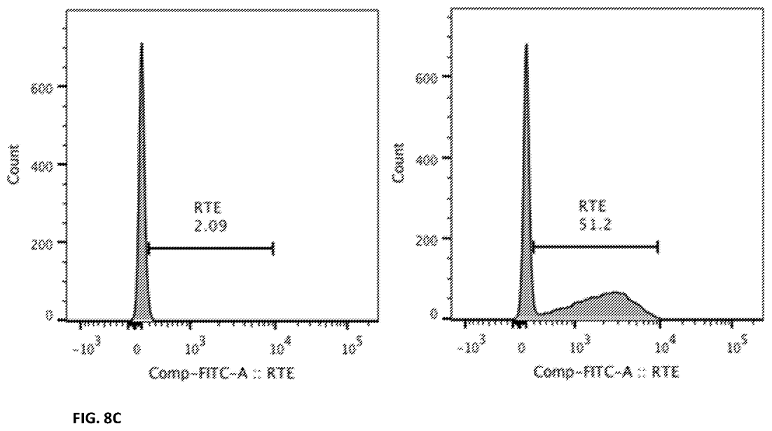

To examine plasmacytoid T cells (PTC) and donor expanded Tregs in Major histocompatibility complex (MHC) mismatched hematopoietic stem cell transplantation (HSCT), irradiated RAG2p-GFP (B6)=BM donor (kb, CD45.2) and B6FIR=T (sp) cell donor (kb, CD45.2) BALB/c (kd, CD45.2) were used. Recipients, Bone marrow and Spleen received PTC treatment, Donor Expanded Tregs, BM transplant only and GVHD Control (see FIGS. 6A, 6B, 7A, 7B, 7C, 8A, 8B, and 8C).

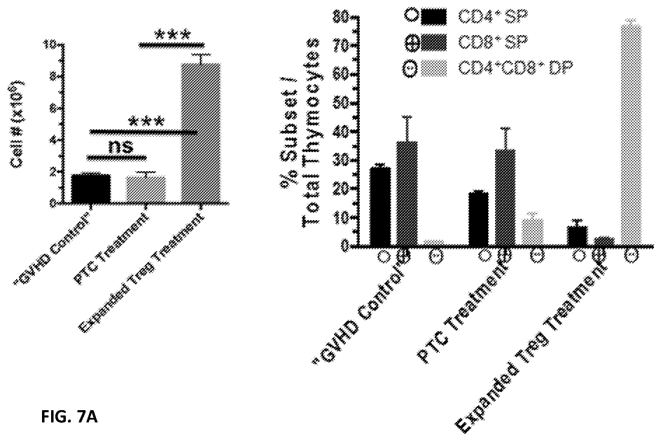

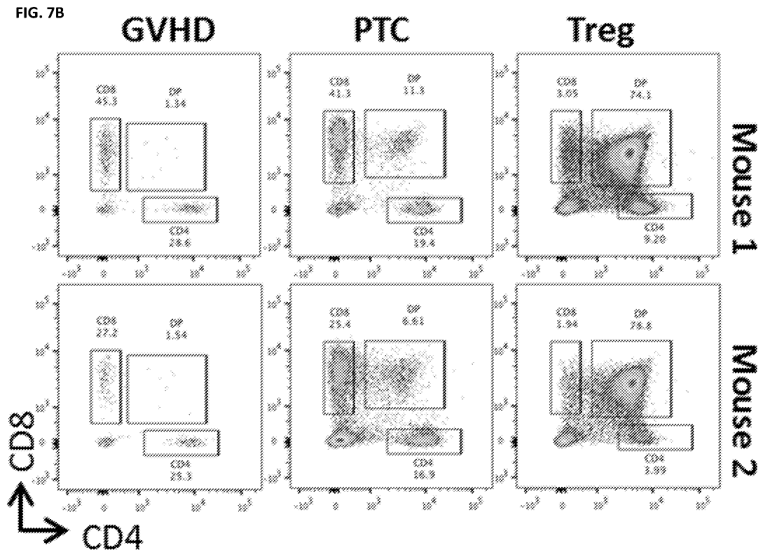

As shown in FIGS. 6A and 6B, immune engraftment following MHC-mismatched HSCT was shown to be more rapid in recipients treated with expanded Tregs when compared to plasmacytoid T cells (PTC). Less thymic damage was observed in recipients of Treg expanded when compared to PTC early post-HSCT (see FIGS. 7A-7C). Further, higher levels of RTE present following MHC-mismatched HSCT using donor cells containing expanded Tregs when compared to normal donor cells and PTC treatment.

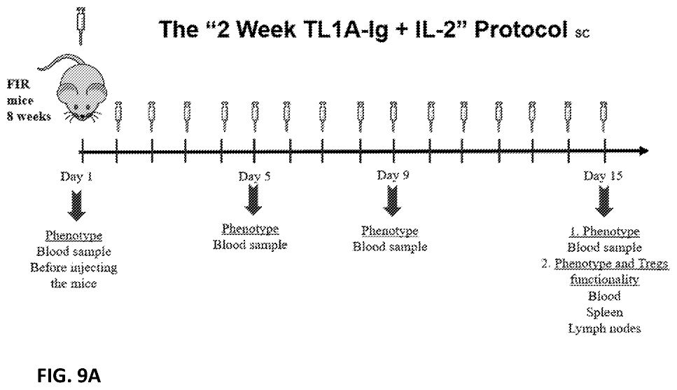

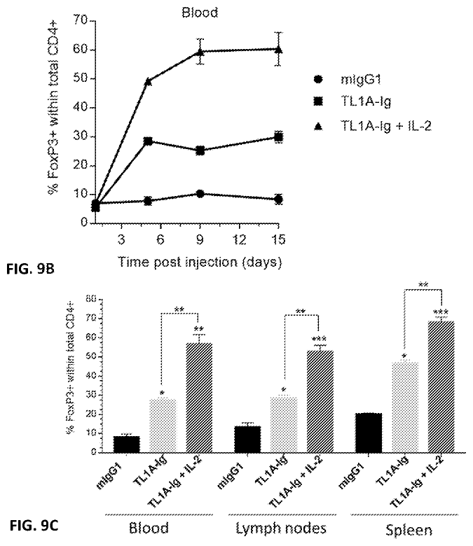

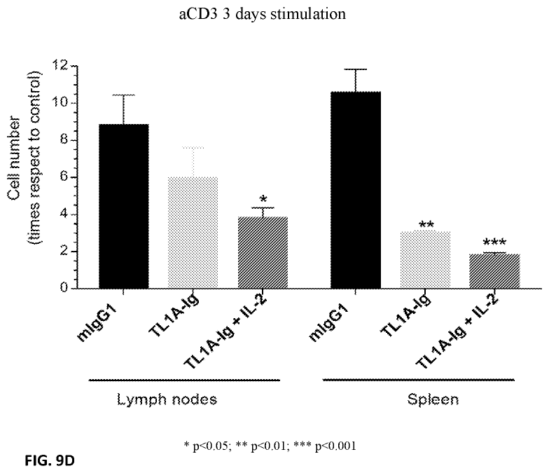

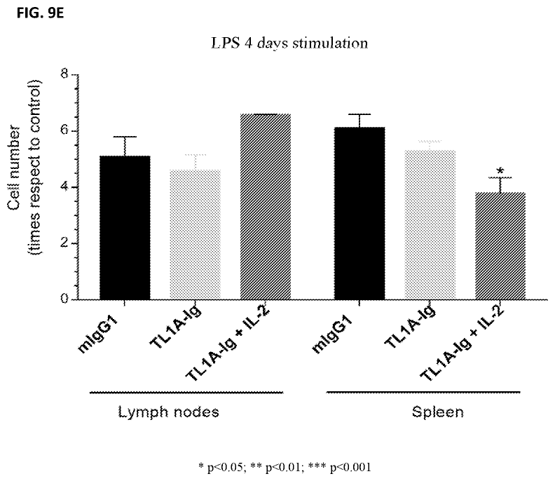

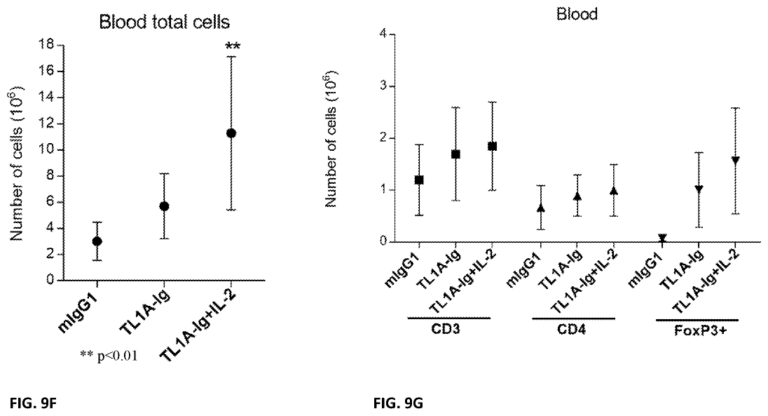

To examine the ability of TL1A-Ig+IL-2 Foxp3-IRES-mRFP (FIR) reporter mouse to monitor Treg activity, a 2-week experimental protocol was designed as shown in FIG. 9A. Eight-weeks old FIR mice (Control (mIgG1), mTL1A, mTL1A+IL-2), received injections, intraperitoneally daily, [mTL1A-Ig]=25 μg/mouse and [IL-2]=10.000 U/mouse. Mice blood samples were collected on day 1, day 5, day 9, and day 15, and flow cytometry was used to analyze cell populations phenotype for CD3, CD4, CD8, T regs (FIR). As shown in FIG. 9B, illustrating the percentage of Foxp3+ CD4+ cells within the total CD4+ cells in blood following mIgG1, TLA-Ig, and TL1A-Ig+IL-2 treatment in days post injection, the TL1A-Ig+IL-2 treatment resulted in the sharp increase in the percentage of Foxp3+ CD4+ cells within the total CD4+ cells in blood post-injection, reaching about 50% on day 5, and the percentage of Foxp3+ CD4+ cells remained high (at least 60%) throughout the experiment until day 15. Thus, the sustained increase in the percentage of Foxp3+ CD4+ cells within the total CD4+ cells was observed post-injection. The time-dependent distribution of the percentage of Foxp3+ CD4+ cells within the total CD4+ cells following the TL1A-Ig treatment alone also exhibited the sustained increase, but the highest percentage of Foxp3+ CD4+ cells observed at days 5 and 15, about 30%, was significantly lower than that observed in the TL1A-Ig+IL-2 treatment. Both TL1A-Ig and TL1A-Ig+IL-2 treatments resulted in the markedly increased percentages of Tregs as compared to control (mIgG1). As shown in FIGS. 9D and 9E, suppressive activity was examined following stimulation with aCD3 for 3 days and stimulation with LPS for 4 days, respectively. FIG. 9F illustrates that the TL1A-Ig+IL-2 treatment resulted in the prominent increase in the total blood cells number, as compared to the number of total blood cells following mIgG1 (control) and TL1A-Ig treatments (where the TL1A-Ig resulted in the cell number increase as compared to the control). FIG. 9G shows the increase in the blood cells number following CD3, CD4, and Foxp3+ cells stimulation under the TL1A-Ig+IL-2 treatment, as compared to respective CD3, CD4, and Foxp3+ cells stimulation with mIgG1 (control) and TL1A-Ig treatments.

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method of treating or preventing graft-versus-host disease (GVHD) in a transplant recipient, comprising administering a human tumor necrosis factor (TNF)-like cytokine 1A (TL1A)-Ig fusion protein to a transplant donor, wherein:
the fusion protein comprises (a) a first polypeptide comprising an extracellular domain of a human TL1A polypeptide or a fragment thereof that specifically binds to Tumor Necrosis Factor Receptor Superfamily, Member 25 (TNFRSF25); and (b) a second polypeptide comprising an immunoglobulin (Ig) polypeptide;
the administration occurs at least 3 times; and
the transplant comprises regulatory T cells (Tregs) from the transplant donor.

2. The method of claim 1, wherein the graft versus host disease is reduced.

3. The method of claim 1, wherein the transplant comprises donor hematopoietic cells.

4. The method of any one of claim 1, wherein the transplant comprises donor stem cells.

5. The method of any one of claim 1, wherein the transplant comprises donor bone marrow cells.

6. The method of any one of claims 1-5, wherein the graft versus host disease is acute graft-versus-host-disease (aGVHD).

7. The method of any one of claims 1-6, wherein the graft versus host disease is chronic graft-versus-host-disease (cGVHD).

8. The method of any one of claims 1-7, wherein the administration to the transplant donor occurs prior to transplant.

9. The method of any one of claims 1-8, wherein the administration is also to the transplant recipient.

10. The method of any one of claims 1-9, wherein the administration to the transplant recipient occurs after the transplant.

11. The method of any one of claims 1-10, wherein the administration is to both the transplant donor and transplant recipient.

12. The method of any one of claims 1-11, wherein the fusion protein causes a sustained increase in Treg cells in the transplant donor and/or transplant recipient.

13. The method of any one of claims 1-11, wherein the fusion protein does not cause substantial Treg suppression in the transplant donor and/or transplant recipient.

14. The method of any one of claims 1-11, wherein the fusion protein does not cause substantial Treg anergy in the transplant donor and/or transplant recipient.

15. The method of any one of claims 1-14, wherein the administration occurs at least 7 times.

16. The method of any one of claims 1-14, wherein the administration occurs at least 10 times.

17. The method of any one of claims 1-14, wherein the administration occurs at least 14 times.

18. The method of any one of claims 1-14, wherein the administration occurs about 3-7 times.

19. The method of any one of claims 1-14, wherein the administration occurs about 3-14 times.

20. The method of any one of claims 1-14, wherein the administration occurs about 3-21 times.

21. The method of any one of claims 1-14, wherein the administration occurs about 3 times.

22. The method of any one of claims 1-14, wherein the administration occurs about 7 times.

23. The method of any one of claims 1-14, wherein the administration occurs about 10 times.

24. The method of any one of claims 1-14, wherein the administration occurs about 14 times.

25. The method of any one of claims 1-14, wherein the administration occurs daily.

26. The method of any one of claims 1-14, wherein the administration occurs twice daily.

27. The method of any one of claims 1-14, wherein the administration occurs daily for 3-7 days.

28. The method of any one of claims 1-14, wherein the administration occurs daily for 7-14 days.

29. The method of any one of claims 1-14, wherein the administration occurs daily for 7-21 days.

30. The method of any one of claims 1-14, wherein the administration occurs daily for at least 7 days.

31. The method of any one of claims 1-14, wherein the administration occurs daily for at least 10 days.

32. The method of any one of claims 1-14, wherein the administration occurs daily for at least 21 days.

33. The method of any one of claims 1-14, wherein the administration occurs before the transplant.

34. The method of any one of claims 1-14, wherein the administration occurs concurrently with the transplant.

35. The method of any one of claims 1-14, wherein the administration occurs after the solid organ transplant.

36. The method of any one of claims 1-14, wherein the administration occurs before and after the solid organ transplant.

37. The method of any one of claims 1-36, wherein the first polypeptide comprises (a) the amino acid sequence of SEQ ID NO: 1, or (b) an amino acid sequence that has at least 95% sequence identity to SEQ ID NO: 1.

38. The method of any one of claims 1-36, wherein the Ig polypeptide comprises one or more of a hinge region, a CH2 domain, and a CH3 domain of an IgG polypeptide.

39. The method of any one of claims 1-36, wherein the Ig polypeptide comprises one or more of a hinge region, a CH2 domain, and a CH3 domain of a human IgG1.

40. The method of any one of claims 1-36, wherein the Ig polypeptide comprises one or more of a hinge region, a CH2 domain, and a CH3 domain of a human IgG2.

41. The method of any one of claims 1-36, wherein the Ig polypeptide comprises all of a hinge region, CH2 domain, and CH3 domain of an IgG polypeptide.

42. The method of any one of claims 1-36, wherein the Ig polypeptide comprises all of a hinge region, CH2 domain, and CH3 domain of a human IgG1.

43. The method of any one of claims 1-36, wherein the Ig polypeptide comprises (a) the amino acid sequence of SEQ ID NO: 2, or (b) an amino acid sequence that has at least 95% sequence identity to SEQ ID NO: 2.

44. The method of any one of claims 1-36, wherein the Ig polypeptide comprises all of a hinge region, CH2 domain, and CH3 domain of a human IgG2.

45. The method of any one of claims 1-36, wherein the Ig polypeptide comprises (a) the amino acid sequence of SEQ ID NO: 4, or (b) an amino acid sequence that has at least 95% sequence identity to SEQ ID NO: 4.

46. The method of any one of claims 1-36, wherein the fusion protein comprises:
(a) the amino acid sequence of SEQ ID NO: 1, or an amino acid sequence that has at least 95% sequence identity to SEQ ID NO: 1; and
(b) the amino acid sequence of SEQ ID NO: 2 or SEQ ID NO: 4, or an amino acid sequence that has at least 95% sequence identity to SEQ ID NO: 2 or SEQ ID NO: 4.

47. The method of any one of claims 1-46, further comprising administering interleukin-2 (IL-2).

48. The method of claim 47, wherein the IL-2 is a low dose of IL-2.

49. The method of claim 48, wherein the low dose of IL-2 is less than 1 million units per square meter per day.

50. The method of claim 49, wherein the low dose of IL-2 is an amount in the range of about 30,000 to about 300,000 units per square meter per day.

51. The method of claim 49, wherein the low dose of IL-2 is about 300,000 units per square meter per day.

52. The method of claim 49, wherein the low dose of IL-2 is about 30,000 units per square meter per day.

53. The method of claim 49, wherein the administration of low dose IL-2 is sequential with the fusion protein.

54. The method of claim 49, wherein the administration of low dose IL-2 is concurrent with the fusion protein.

55. A method of reducing or preventing an immune response in a solid organ transplant recipient, comprising administering a human TL1A-Ig fusion protein to the solid organ transplant recipient, wherein:
the fusion protein comprises (a) a first polypeptide comprising an extracellular domain of a human TL1A polypeptide or a fragment thereof that specifically binds to TNFRSF25; and (b) a second polypeptide comprising an immunoglobulin (Ig) polypeptide;
the administration occurs at least 3 times; and
the immune response is a rejection of the solid organ transplant.

56. The method of claim 55, wherein the method prevents a solid organ transplant rejection.

57. The method of claim 55, wherein the method reduces the likelihood of solid organ transplant rejection.

58. The method of any one of claims 55-57, wherein the fusion protein causes a sustained increase in Treg cells in the solid organ transplant recipient.

59. The method of claim 58, wherein the sustained increase in Treg cells comprises a substantially similar level of Treg cells in the solid organ transplant recipient after the first and last fusion protein administration.

60. The method of any one of claims 55-59, wherein the fusion protein does not cause substantial Treg suppression in the transplant donor and/or transplant recipient.

61. The method of any one of claims 55-59, wherein the fusion protein does not cause substantial Treg anergy in the transplant donor and/or transplant recipient.

62. The method of any one of claims 55-61, wherein the solid organ is selected from lung, kidney, heart, liver, pancreas, thymus, gastrointestinal tract, cornea, eye, and composite allografts.

63. The method of any one of claims 55-61, wherein the administration occurs at least 7 times.

64. The method of any one of claims 55-61, wherein the administration occurs at least 10 times.

65. The method of any one of claims 55-61, wherein the administration occurs at least 14 times.

66. The method of any one of claims 55-61, wherein the administration occurs about 3-7 times.

67. The method of any one of claims 55-61, wherein the administration occurs about 3-14 times.

68. The method of any one of claims 55-61, wherein the administration occurs about 3-21 times.

69. The method of any one of claims 55-61, wherein the administration occurs about 3 times.

70. The method of any one of claims 55-61, wherein the administration occurs about 7 times.

71. The method of any one of claims 55-61, wherein the administration occurs about 10 times.

72. The method of any one of claims 55-61, wherein the administration occurs about 14 times.

73. The method of any one of claims 55-61, wherein the administration occurs daily.

74. The method of any one of claims 55-61, wherein the administration occurs twice daily.

75. The method of any one of claims 55-61, wherein the administration occurs daily for 3-7 days.

76. The method of any one of claims 55-61, wherein the administration occurs daily for 7-14 days.

77. The method of any one of claims 55-61, wherein the administration occurs daily for 7-21 days.

78. The method of any one of claims 55-61, wherein the administration occurs daily for at least 7 days.

79. The method of any one of claims 55-61, wherein the administration occurs daily for at least 10 days.

80. The method of any one of claims 55-61, wherein the administration occurs daily for at least 21 da

81. The method of any one of claims 55-61, wherein the administration occurs before the solid organ transplant

82. The method of any one of claims 55-81, wherein the administration occurs concurrently with the solid organ transplant.

83. The method of any one of claims 55-81, wherein the administration occurs after the solid organ transplant.

84. The method of any one of claims 55-81, wherein the administration occurs before and after the solid organ transplant.

85. The method of any one of claims 55-84, wherein the first polypeptide comprises (a) the amino acid sequence of SEQ ID NO: 1, or (b) an amino acid sequence that has at least 95% sequence identity to SEQ ID NO: 1.

86. The method of any one of claims 55-84, wherein the Ig polypeptide comprises one or more of a hinge region, a CH2 domain, and a CH3 domain of an IgG polypeptide.

87. The method of any one of claims 55-84, wherein the Ig polypeptide comprises one or more of a hinge region, a CH2 domain, and a CH3 domain of a human IgG1.

88. The method of any one of claims 55-84, wherein the Ig polypeptide comprises one or more of a hinge region, a CH2 domain, and a CH3 domain of a human IgG2.

89. The method of any one of claims 55-84, wherein the Ig polypeptide comprises all of a hinge region, CH2 domain, and CH3 domain of an IgG polypeptide.

90. The method of any one of claims 55-84, wherein the Ig polypeptide comprises all of a hinge region, CH2 domain, and CH3 domain of a human IgG1.

91. The method of any one of claims 55-84, wherein the Ig polypeptide comprises (a) the amino acid sequence of SEQ ID NO: 2, or (b) an amino acid sequence that has at least 95% sequence identity to SEQ ID NO: 2.

92. The method of any one of claims 55-84, wherein the Ig polypeptide comprises all of a hinge region, CH2 domain, and CH3 domain of a human IgG2.

93. The method of any one of claims 55-84, wherein the Ig polypeptide comprises (a) the amino acid sequence of SEQ ID NO: 4, or (b) an amino acid sequence that has at least 95% sequence identity to SEQ ID NO: 4.

94. The method of any one of claims 55-84, wherein the fusion protein comprises:
(a) the amino acid sequence of SEQ ID NO: 1, or an amino acid sequence that has at least 95% sequence identity to SEQ ID NO: 1 and
(b) the amino acid sequence of SEQ ID NO: 2 or SEQ ID NO: 4, or an amino acid sequence that has at least 95% sequence identity to SEQ ID NO: 2 or SEQ ID NO: 4.

95. The method of any one of claims 55-94, further comprising administering IL-2.

96. The method of claim 95, wherein the IL-2 is a low dose of IL-2.

97. The method of claim 96, wherein the low dose of IL-2 is less than 1 million units per square meter per day.

98. The method of claim 97, wherein the low dose of IL-2 is an amount in the range of about 30,000 to about 300,000 units per square meter per day.

99. The method of claim 97, wherein the low dose of IL-2 is about 300,000 units per square meter per day.

100. The method of claim 97, wherein the low dose of IL-2 is about 30,000 units per square meter per day.

101. The method of any one of claims 96-100, wherein the administration of low dose IL-2 is sequential with the fusion protein.

102. The method of any one of claims 96-100, wherein the administration of low dose IL-2 is concurrent with the fusion protein.

103. A method for generating a sustained amount of Tregs in vivo, comprising administering to a subject in need thereof:
a human TL1A-Ig fusion protein, the fusion protein comprising (a) a first polypeptide comprising an extracellular domain of a human TL1A polypeptide or a fragment thereof that specifically binds to TNFRSF25; and (b) a second polypeptide comprising an immunoglobulin (Ig) polypeptide to expand and selectively activate a population of Tregs,
wherein the administration occurs at least 3 times.

104. The method of claim 103, further comprising administering a low dose of IL-2.

105. The method of claim 104, wherein the low dose of IL-2 is less than 1 million units per square meter per day.

106. The method of claim 105, wherein the low dose of IL-2 is an amount in the range of about 30,000 to about 300,000 units per square meter per day.

107. The method of claim 105, wherein the low dose of IL-2 is about 300,000 units per square meter per day.

108. The method of claim 105, wherein the low dose of IL-2 is about 30,000 units per square meter per day.

109. A method for generating Tregs ex vivo, comprising:
(a) isolating a population of Tregs from a subject; and
(b) contacting the isolated population of Tregs with a human TL1A-Ig fusion protein, the fusion protein comprising (a) a first polypeptide comprising an extracellular domain of a human TL1A polypeptide or a fragment thereof that specifically binds to TNFRSF25; and (b) a second polypeptide comprising an immunoglobulin (Ig) polypeptide, to expand and selectively activate a population of Tregs.

110. The method of claim 109, further comprising contacting the Tregs with a low dose of IL-2.

111. The method of claim 110, wherein the low dose of IL-2 is less than 1 million units per square meter per day.

112. The method of claim 111, wherein the low dose of IL-2 is an amount in the range of about 30,000 to about 300,000 units per square meter per day.

113. The method of claim 111, wherein the low dose of IL-2 is about 300,000 units per square meter per day.

114. The method of claim 111, wherein the low dose of IL-2 is about 30,000 units per square meter per day.

115. The method of any one of claims 103-114, wherein the Tregs are characterized by significantly fewer naive Tregs and increased central memory (CD62-L^{hi}CD44+) and effector/memory (CD62-L^{lo}CD44+) Tregs in the spleen and peripheral lymph nodes (pLN) as compared to untreated Tregs.

116. The method of any one of claims 103-114, wherein the Tregs are characterized by a significant decrease in Ly6C expression as compared to untreated Tregs.

117. The method of any one of claims 103-114, wherein the Tregs are characterized by higher levels of Treg effector molecules and mediated enhanced in vitro suppressor activity as compared to untreated Tregs.

118. The method of any one of claims 103-114, wherein the Tregs are characterized by higher levels of activation and functional markers in the peripheral lymph nodes and the spleen as compared to untreated Tregs, the activation and functional markers selected from CD39, Nrp1, ICOS, CD73, KLRG1, CD103, Annexin V, PD-1 and CTLA-4.

119. A method for preventing, reducing or ameliorating an autoimmune disease or disorder in a subject in need thereof comprising:
administering a human TL1A-Ig fusion protein to the subject, the fusion protein comprising
(a) a first polypeptide comprising an extracellular domain of a human TL1A polypeptide or a fragment thereof that specifically binds to TNFRSF25; and
(b) a second polypeptide comprising an immunoglobulin (Ig) polypeptide,
wherein the administration occurs at least 3 times.

120. The method of claim 119, further comprising administering a low dose of IL-2.

121. The method of claim 120, wherein the low dose of IL-2 is less than 1 million units per square meter per day.

122. The method of claim 121, wherein the low dose of IL-2 is an amount in the range of about 30,000 to about 300,000 units per square meter per day.

123. The method of claim 121, wherein the low dose of IL-2 is about 300,000 units per square meter per day.

124. The method of claim 121, wherein the low dose of IL-2 is about 30,000 units per square meter per day.

125. A method for preventing, reducing or ameliorating an autoimmune disease or disorder in a subject in need thereof comprising administering a population of Tregs to the subject, wherein the population of Tregs has been treated with a human TL1A-Ig fusion protein, the fusion protein comprising
(a) a first polypeptide comprising an extracellular domain of a human TL1A polypeptide or a fragment thereof that specifically binds to TNFRSF25; and
(b) a second polypeptide comprising an immunoglobulin (Ig) polypeptide.

126. The method of claim 125, wherein the population of Tregs has been further treated with a low dose of IL-2.

127. The method of claim 126, wherein the low dose of IL-2 is less than 1 million units per square meter per day.

128. The method of claim 127, wherein the low dose of IL-2 is an amount in the range of about 30,000 to about 300,000 units per square meter per day.

129. The method of claim 127, wherein the low dose of IL-2 is about 300,000 units per square meter per day.

130. The method of claim 127, wherein the low dose of IL-2 is about 30,000 units per square meter per day.

131. The method of claim 125, wherein the Tregs are administered at lower doses than a treatment with untreated Tregs.

132. The method of any one of claims 103-131, wherein the first polypeptide comprises (a) the amino acid sequence of SEQ ID NO: 1, or (b) an amino acid sequence that has at least 95% sequence identity to SEQ ID NO: 1.

133. The method of any one of claims 103-131, wherein the Ig polypeptide comprises one or more of a hinge region, a CH2 domain, and a CH3 domain of an IgG polypeptide.

134. The method of any one of claims 103-131, wherein the Ig polypeptide comprises one or more of a hinge region, a CH2 domain, and a CH3 domain of a human IgG1.

135. The method of any one of claims 103-131, wherein the Ig polypeptide comprises one or more of a hinge region, a CH2 domain, and a CH3 domain of a human IgG2.

136. The method of any one of claims 103-131, wherein the Ig polypeptide comprises all of a hinge region, CH2 domain, and CH3 domain of an IgG polypeptide.

137. The method of any one of claims 103-131, wherein the Ig polypeptide comprises all of a hinge region, CH2 domain, and CH3 domain of a human IgG1.

138. The method of any one of claims 103-131, wherein the Ig polypeptide comprises (a) the amino acid sequence of SEQ ID NO: 2, or (b) an amino acid sequence that has at least 95% sequence identity to SEQ ID NO: 2.

139. The method of any one of claims 103-131, wherein the Ig polypeptide comprises all of a hinge region, CH2 domain, and CH3 domain of a human IgG2.

140. The method of any one of claims 103-131, wherein the Ig polypeptide comprises (a) the amino acid sequence of SEQ ID NO: 4, or (b) an amino acid sequence that has at least 95% sequence identity to SEQ ID NO: 4.

141. The method of any one of claims 103-131, wherein the fusion protein comprises:
(a) the amino acid sequence of SEQ ID NO: 1, or an amino acid sequence that has at least 95% sequence identity to SEQ ID NO: 1 and
(b) the amino acid sequence of SEQ ID NO: 2 or SEQ ID NO: 4, or an amino acid sequence that has at least 95% sequence identity to SEQ ID NO: 2 or SEQ ID NO: 4.


SEQUENCE LISTING (4 sequences)

SEQ ID NO: 1 - protein, 192 aa, Homo sapiens
RAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 2 - protein, 228 aa, Homo sapiens
CDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 422 aa, Homo sapiens
CDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKEFRAQGEACVQFQALKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 4 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK